(19) United States
(12) Patent Application Publication
(10) Pub. No.: US 2006/0111438 A1
(43) Pub. Date: May 25, 2006

(54) Asymmetric synthesis of substituted dihydrobenzofurans

(75) Inventors: Alexander V. Gontcharov, Rivervale, NJ (US); Gulnaz Khafizova, West Nyack, NY (US); John R. Potoski, West Nyack, NY (US); Qing Yu, Laval (CA); Chia-Cheng Shaw, Montreal (CA); Gary Paul Stack, Ambler, PA (US); Dahui Zhou, New Brunswick, NJ (US)

Correspondence Address: CHOATE, HALL & STEWART LLP, PATENT GROUP, TWO INTERNATIONAL PLACE, BOSTON, MA, 02110, US

(73) Assignee: Wyeth, Madison, NJ (US)

(21) Appl. No.: 11/255,645

(22) Filed: Oct. 21, 2005

Related U.S. Application Data
(60) Provisional application No. 60/621,024, filed on Oct. 21, 2004.
(60) Provisional application No. 60/674,177, filed on Apr. 22, 2005.
(60) Provisional application No. 60/721,064, filed on Sep. 28, 2005.

(51) Int. Cl. A61K 31/343; C07D 307/87; C07F 3/02

(52) U.S. Cl. 514/469; 549/467; 260/665 G

(57) ABSTRACT

The present invention concerns production of a compound of the formula 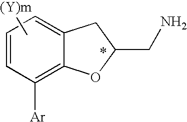
or a pharmaceutically acceptable salt thereof by a process which utilizes the cyclization of a compound of the formula: 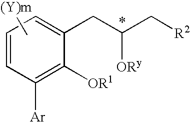
where Ar, Y, R¹, R^{y}, R², and m are as defined herein.

### RELATED APPLICATIONS

This application is claims priority to U.S. provisional applications Ser. No. 60/621,024, filed Oct. 21, 2004, Ser. No. 60/674,177 filed Apr. 22, 2005, and Ser. No. 60/721,064, filed Sep. 28, 2005, the entire contents of each of which are hereby incorporated herein by reference.

### FIELD OF THE INVENTION

The present invention relates to the asymmetric synthesis of substituted dihydrobenzofurans.

### BACKGROUND OF THE INVENTION

Schizophrenia affects approximately 5 million people. The most prevalent treatments for schizophrenia are currently the ‘atypical’ antipsychotics, which combine dopamine (D₂) and serotonin (5-HT_{2A}) receptor antagonism. Despite the reported improvements in efficacy and side-effect liability of atypical antipsychotics relative to typical antipsychotics, these compounds do not appear to adequately treat all the symptoms of schizophrenia and are accompanied by problematic side effects, such as weight gain (Allison, D. B., et. al., Am. J. Psychiatry, 156: 1686-1696, 1999; Masand, P. S., Exp. Opin. Pharmacother. 1: 377-389, 2000; Whitaker, R., Spectrum Life Sciences. Decision Resources. 2:1-9, 2000).

Atypical antipsychotics also bind with high affinity to 5-HT_{2C} receptors and function as 5-HT_{2C} receptor antagonists or inverse agonists. Weight gain is a problematic side effect associated with atypical antipsychotics such as clozapine and olanzapine, and it has been suggested that 5-HT_{2C} antagonism is responsible for the increased weight gain. Conversely, stimulation of the 5-HT_{2C} receptor is known to result in decreased food intake and body weight (Walsh et. al., Psychopharmacology 124: 57-73, 1996; Cowen, P. J., et. al., Human Psychopharmacology 10: 385-391, 1995; Rosenzweig-Lipson, S., et. al., ASPET abstract, 2000).

Several lines of evidence support a role for 5-HT_{2C} receptor agonism or partial agonism as a treatment for schizophrenia. Studies suggest that 5-HT_{2C} antagonists increase synaptic levels of dopamine and may be effective in animal models of Parkinson's disease (Di Matteo, V., et. al., Neuropharmacology 37: 265-272, 1998; Fox, S. H., et. al., Experimental Neurology 151: 35-49, 1998). Since the positive symptoms of schizophrenia are associated with increased levels of dopamine, compounds with actions opposite to those of 5-HT_{2C} antagonists, such as 5-HT_{2C} agonists and partial agonists, should reduce levels of synaptic dopamine. Recent studies have demonstrated that 5-HT_{2C} agonists decrease levels of dopamine in the prefrontal cortex and nucleus accumbens (Millan, M. J., et. al., Neuropharmacology 37: 953-955, 1998; Di Matteo, V., et. al., Neuropharmacology 38: 1195-1205, 1999; Di Giovanni, G., et. al., Synapse 35: 53-61, 2000), brain regions that are thought to mediate critical antipsychotic effects of drugs like clozapine. However, 5-HT_{2C} agonists do not decrease dopamine levels in the striatum, the brain region most closely associated with extrapyramidal side effects. In addition, a recent study demonstrates that 5-HT_{2C} agonists decrease firing in the ventral tegmental area (VTA), but not in the substantia nigra. The differential effects of 5-HT_{2C} agonists in the mesolimbic pathway relative to the nigrostriatal pathway suggest that 5-HT_{2C} agonists have limbic selectivity, and will be less likely to produce extrapyramidal side effects associated with typical antipsychotics.

### SUMMARY OF THE INVENTION

As described herein, the present invention provides methods for preparing compounds having activity as 5HT_{2C} agonists or partial agonists. These compounds are useful for treating disorders including schizophrenia, schizophreniform disorder, schizoaffective disorder, delusional disorder, substance-induced psychotic disorder, L-DOPA-induced psychosis, psychosis associated with Alzheimer's dementia, psychosis associated with Parkinson's disease, psychosis associated with Lewy body disease, dementia, memory deficit, intellectual deficit associated with Alzheimer's disease, bipolar disorders, depressive disorders, mood episodes, anxiety disorders, adjustment disorders, eating disorders, epilepsy, sleep disorders, migraines, sexual dysfunction, gastrointestinal disorders, obesity, or a central nervous system deficiency associated with trauma, stroke, or spinal cord injury. Such compounds include those of formula II: 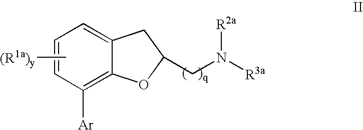
or a pharmaceutically acceptable salt thereof, wherein each of R^{1a}, R^{2a}, R^{3a}, Ar, q, and y is as defined herein.

The present invention also provides synthetic intermediates useful for preparing such compounds.

### DETAILED DESCRIPTION OF CERTAIN EMBODIMENTS

The methods and intermediates of the present invention are useful for preparing compounds as described in, e.g. U.S. patent application entitled “Dihydrobenzofuranyl Alkanamine Derivatives and Methods for Using Same,” filed in the name of Jonathan Gross, et al., having Ser. No. 11/113,170, filed Apr. 22, 2005, and claiming benefit to U.S. application Ser. No. 10/970,014, filed Oct. 21, 2004, and U.S. provisional application 60/514,454, filed on Oct. 24, 2003, each of which is hereby incorporated herein by reference in its entirety for all purposes. In certain embodiments, the present compounds are generally prepared according to Scheme I set forth below:

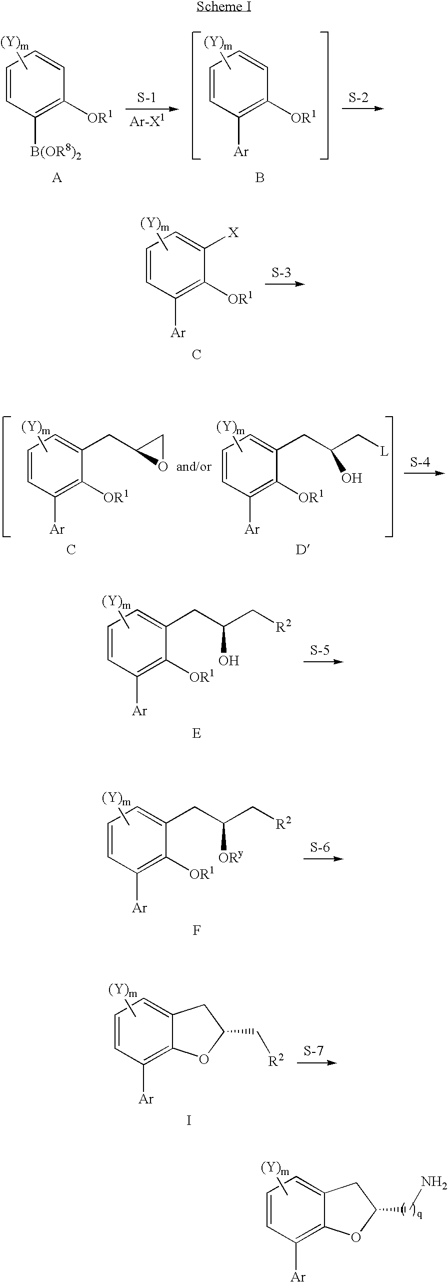

In Scheme I above, each of R¹, R², R^{y}, Ar, Y, R⁸, X, X¹, q, and m is as defined below and in classes and subclasses as described herein.

It will be appreciated that although Scheme I depicts a method for preparing a specific enantiomer of formula I, the opposite enantiomer is similarly prepared using the appropriate glycidyl ether.

In one aspect, the present invention provides methods for preparing a chiral non-racemic biaryl compound of formula D according to the steps depicted in Scheme I, above. Catalyst and reaction conditions for the Suzuki reaction of step S-1 above are well known in the art. See, for example, Miyaura, N.; Suzuki, A. Chem. Rev. 1995, 95, 2457. In certain embodiments, the Suzuki coupling at step S-1 is performed in the presence of a palladium containing compound. In other embodiments, the palladium containing compound is Pd(PPh₃)₄. The reaction may also be performed in the presence of a base. In certain embodiments, the base is MOH, M₂CO₃, or M₃PO₄, wherein each M is independently an alkali metal. In some embodiments, the alkali metal is sodium, potassium, or lithium. In other embodiments, step S-1 is performed in the presence of NaOH. The reaction is optionally carried out in the presence of a solvent. Suitable solvents for the coupling at step S-1 include dimethoxyethane, toluene, ethanol, isopropanol, methanol, and tetrahydrofuran (THF). In certain embodiments, the suitable solvent for the coupling step of S-1 includes water. In certain embodiments, the Suzuki coupling reaction is carried out at a temperature in the range of about 20° C. to about the temperature sufficient to reflux the solvent or mixture thereof. In other embodiments, the coupling reaction is carried out at about 60° C. to about 90° C. In still other embodiments, the coupling reaction is carried out at about 70° C. to about 80° C.

As depicted in Scheme I above, a compound of formula B is halogenated at step S-2 to form a compound of formula C wherein X is halogen. One of ordinary skill in the art would recognize that a variety of halogenating agents are suitable for preparing a compound of formula C from a compound of formula B. In certain embodiments, X is bromo and the halogenating agent used at step S-2 is bromine. In other embodiments, X is bromo and the halogenating agent used at step S-2 is a compound containing an N—Br group (e.g., N-bromosuccinimide). Other brominating agents are known to those skilled in the art. In certain embodiments, one or more additives are used in the halogenation reaction. These additives include inorganic acid such as H₂SO₄ (used, for example, with an N—Br brominating agent), Lewis acid, or AcONa (used, for example, with bromine). In other embodiments, the halogenation at step S-2 is performed in a suitable solvent. Suitable solvents for the halogenation at step S-2 include protic solvents, ethers, chlorinated hydrocarbons, and mixtures thereof. Such suitable solvents include dioxane, THF, acetic acid, CH₂Cl₂, CHCl₃, CCl₄, dichloroethane, and the like. In some embodiments, the reaction is performed at a temperature of about 18° C. to about the temperature sufficient to reflux the solvent. In other embodiments, the additive is p-toluenesulfonic acid and the solvent is acetic acid or formic acid.

At step S-3, the halogenated compound of formula C is treated with a suitable Grignard reagent or magnesium metal then a chiral non-racemic epoxide of the formula: 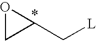
wherein L is a suitable leaving group. In other embodiments, said reagent is of formula RMgX², wherein X² is halogen and R is an alkyl group.

As defined above, L is a suitable leaving group. Suitable leaving groups are well known in the art, e.g., see, “Advanced Organic Chemistry,” Jerry March, 5^{th} Ed., pp. 445-448, John Wiley and Sons, N.Y. Such leaving groups include, but are not limited to, halogen, alkoxy, sulfonyloxy, optionally substituted alkylsulfonyloxy, optionally substituted alkenylsulfonyloxy, optionally substituted arylsulfonyloxy. Examples of suitable leaving groups include chloro, iodo, bromo, fluoro, methanesulfonyloxy (mesyloxy), tosyloxy, triflyloxy, nitrophenylsulfonyloxy (nosyloxy), and bromophenylsulfonyloxy (brosyloxy). In certain embodiments, L is halogen. In other embodiments, L is an optionally substituted alkylsulfonyloxy, optionally substituted alkenylsulfonyloxy, or optionally substituted arylsulfonyloxy group.

At step S-4, a compound of formula D or D′ or a mixture of the two is treated with a suitable reagent to form a compound of formula E. In certain embodiments, a compound of formula D and/or D′ is treated with a reagent containing a suitably protected amino group to form a compound of formula E wherein R² is a protected amino group. In other embodiments, a compound of formula D and/or D′ is treated with a suitable cyano reagent to form a compound of formula E wherein R² is CN.

The hydroxyl group of formula E is converted to an OR^{y} moiety at step S-5 to form a compound of formula F. In certain embodiments, said OR^{y} moiety is a suitable leaving group as described herein. In particular, R^{y} may be an organosulfonyl group. At S-6 the compound having formula F is cyclized to form the compound I, where necessary with the use of conditions for cleaving the protecting group R′. Step S-7 includes reduction of an azide or nitrile as explained above or converting a protected amino group as R² into an amino group.

Unless otherwise indicated, the following terms have the following meanings:

The term “alkyl,” as used herein, refers to a hydrocarbon group having 1 to 8 carbon atoms, preferably 1 to 6 carbon atoms, and more preferably 1 to 4 carbon atoms. The term “alkyl” includes, but is not limited to, straight and branched groups such as methyl, ethyl, n-propyl, isopropyl, n-butyl, isobutyl, sec-butyl, t-butyl, n-pentyl, isopentyl, neo-pentyl, n-hexyl, and isohexyl. The term “lower alkyl” refers to an alkyl group having 1 to 4 carbon atoms.

The term “alkenyl,” as used herein refers to a straight or branched hydrocarbon group having 2 to 8 carbon atoms and that contains 1 to 3 double bonds. Examples of alkenyl groups include vinyl, prop-1-enyl, allyl, methallyl, but-1-enyl, but-2-enyl, but-3-enyl, or 3,3-dimethylbut-1-enyl. The term “lower alkenyl” refers to a straight or branched alkenyl group having 1 to 4 carbon atoms.

The term “cycloaliphatic,” as used herein, refers to a saturated or partially unsaturated hydrocarbon monocyclic or bicyclic ring having 3 to 10 carbon atoms and more preferably 5 to 7 carbon atoms. In certain embodiments, the cyclic cycloaliphatic group is bridged. As used herein, the term “bridged” refers to a cycloaliphatic group that contains at least one carbon-carbon bond between two non-adjacent carbon atoms of the cycloalkyl ring. As used herein, the term “partially unsaturated” refers to a nonaromatic cycloaliphatic group containing at least one double bond and, in certain embodiments, only one double bond. In certain embodiments, the cycloaliphatic group is saturated. The cycloaliphatic group may be unsubstituted or substituted as described hereinafter.

The term “alkylcycloaliphatic,” as used herein, refers to the group —(CH₂)ᵣcycloaliphatic, where cycloaliphatic is as defined above and r is 1 to 6, preferably 1 to 4, and more preferably 1 to 3.

The term “heterocycloalkyl,” as used herein, refers to a 3 to 10 membered monocyclic or bicyclic ring having 1-3 heteroatoms independently selected from oxygen, nitrogen, or sulfur. In certain embodiments, heterocycloalkyl refers to a 5 to 7 membered ring having 1-2 heteroatoms independently selected from oxygen, nitrogen, or sulfur. The heterocycloalkyl group may be saturated or partially unsaturated, and may be monocyclic or bicyclic (such as bridged). Preferably, the heterocycloalkyl is monocyclic. The heterocycloalkyl group may be unsubstituted or substituted as described hereinafter.

The term “aryl” used alone or as part of a larger moiety as in “aralkyl”, “aralkoxy”, or “aryloxyalkyl”, refers to monocyclic, bicyclic and tricyclic ring systems having a total of six to fourteen ring members, wherein at least one ring in the system is aromatic and wherein each ring in the system contains 3 to 7 ring members. The term “aryl” may be used interchangeably with the term “aryl ring”. The term “aryloxy,” as used herein, refers to the group —OAr, where Ar is a 6-10 membered aryl group. The term “aralkoxy”, as used herein, refers to a group of the formula —O(CH₂)ᵣAr, wherein r is 1-6. The term “aryloxyalkyl”, as used herein, refers to a group of the formula —(CH₂)ᵣOAr, wherein r is 1-6.

The term “heteroaryl”, used alone or as part of a larger moiety as in “heteroaralkyl” or “heteroarylalkoxy”, refers to monocyclic, bicyclic and tricyclic ring systems having a total of five to fourteen ring members, wherein at least one ring in the system is aromatic, at least one ring in the system contains one or more heteroatoms independently selected from nitrogen, oxygen, or sulfur, and wherein each ring in the system contains 3 to 7 ring members. The term “heteroaryl” may be used interchangeably with the term “heteroaryl ring” or the term “heteroaromatic”. In certain embodiments, such heteroaryl ring systems include furanyl, thienyl, pyrazolyl, imidazolyl, isoxazolyl, oxadiazolyl, oxazolyl, pyrrolyl, pyridyl, pyrimidyl, pyridazinyl, triazinyl, thiazolyl, triazolyl, tetrazolyl, quinolinyl, isoquinolinyl, quinazolinyl, indolinyl, indazolyl, benzothienyl, benzofuranyl, benzisoxazolyl, benzimidazolyl, benzothiazolyl, benzoxazolyl, isoindolyl, and acridinyl, to name but a few.

The term “heteroaralkyl”, as used herein, refers to a group of the formula —(CH₂)ᵣHet, wherein Het is a heteroaryl group as defined above and r is 1-6. The term “heteroarylalkoxy”, as used herein, refers to a group of the formula —O(CH₂)ᵣHet wherein Het is a heteroaryl group as defined above and r is 1-6. Any aryl, heteroaryl, cycloaliphatic or heterocycloalkyl may optionally be substituted with 1 to 5 substituents independently selected from halogen, hydroxyl, cyano, alkyl of 1 to 6 carbon atoms, perfluoroalkyl of 1 to 6 carbon atoms, alkoxy of 1 to 6 carbon atoms, or perfluoroalkoxy of 1 to 6 carbon atoms.

The term “perfluoroalkyl,” as used herein, refers to an alkyl group as defined herein in which all hydrogen atoms are replaced with fluorine.

The term “lower haloalkyl”, as used herein, refers to a C₁₋₄ alkyl group as defined herein in which one or more hydrogen atoms are replaced with a halogen atom.

The term “alkanesulfonamido,” as used herein, refers to the group R—S(O)₂—NH— where R is an alkyl group of 1 to 6 carbon atoms.

The term “alkoxy,” as used herein, refers to the group R—O— where R is an alkyl group of 1 to 6 carbon atoms.

The term “perfluoroalkoxy,” as used herein, refers to the group R—O where R is a perfluoroalkyl group of 1 to 6 carbon atoms.

The terms “monoalkylamino” and “dialkylamino,” as used herein, respectively refer to —NHR and —NR^{a}R^{b}, where R, R^{a} and R^{b} are each an independently selected C₁₋₆ alkyl group.

The terms “halogen” or “halo,” as used herein, refer to chlorine, bromine, fluorine or iodine.

The term “protecting group” such as “hydroxyl protecting group” and “amine protecting group” are well understood by one skilled in the art. In particular one skilled in the art is aware of various protecting groups for use to protect hydroxyl and primary and secondary amine groups. Protecting groups, including include those described for example, in T. W. Greene and P. G. M. Wuts, “Protecting Groups in Organic Synthesis” (1991) provided that they are suitable for use in the chemistries described herein. Particular examples of hydroxyl protecting groups include methyl, benzyl, benzyloxymethyl, or allyl.

Amino protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable amino protecting groups, taken with the —NH— moiety to which it is attached, include, but are not limited to, aralkylamines, carbamates, allyl amines, amides, and the like. Examples of such groups include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, benzyl (Bn), fluorenylmethylcarbonyl (Fmoc), acetyl, chloroacetyl, dichloroacetyl, trichloroacetyl, phenylacetyl, trifluoroacetyl, benzoyl, and the like. In other embodiments, an amino protecting group is acetyl, chloroacetyl, dichloroacetyl, trichloroacetyl, phenylacetyl, or trifluoroacetyl. In still other embodiments, an amino group may be in protected form as a phthalimide or azide.

One of ordinary skill in the art will appreciate that certain moieties are incompatible with (i.e. may interfere with) certain chemical transformations as described herein. Thus, it will be understood that in for certain chemical transformations, certain moieties, e.g. a hydroxyl group or an amino group (primary or secondary), are preferably protected by a suitable protecting group as described herein prior to those transformations. By way of example, one of ordinary skill in the art would recognize that when R² is —NH₂, then that amino group is preferably protected prior to step S-5.

Suitable leaving groups are well known in the art, e.g., see, “Advanced Organic Chemistry,” Jerry March, 5^{th} Ed., pp. 445-448, John Wiley and Sons, N.Y. Such leaving groups include, but are not limited to, halogen, alkoxy, sulfonyloxy, optionally substituted alkylsulfonyloxy, optionally substituted alkenylsulfonyloxy, optionally substituted arylsulfonyloxy. Examples of suitable leaving groups include chloro, iodo, bromo, fluoro, methanesulfonyl (mesyl), tosyl, triflyl, nitrophenylsulfonyl (nosyl), bromophenylsulfonyl (brosyl), and the like.

The compounds of the present invention may contain an asymmetric atom, and some of the compounds may contain one or more asymmetric atoms or centers, which may thus give rise to optical isomers (enantiomers) and diastereomers. In certain embodiments, the asymmetric atom is indicated with a “*”. When shown without respect to the stereochemistry, the present invention includes all optical isomers (enantiomers) and diastereomers (geometric isomers); as well as the racemic and resolved, enantiomerically pure R and S stereoisomers; as well as other mixtures of the R and S stereoisomers and pharmaceutically acceptable salts thereof. Optical isomers may be obtained in pure form by standard procedures known to those skilled in the art, and include, but are not limited to, diastereomeric salt formation, kinetic resolution, and asymmetric synthesis. It is also understood that this invention encompasses all possible regioisomers, and mixtures thereof, which may be obtained in pure form by standard separation procedures known to those skilled in the art, and include, but are not limited to, column chromatography, thin-layer chromatography, and high-performance liquid chromatography. Thus, the compounds of this invention include racemates, enantiomers, or geometric isomers of the compounds shown herein.

It is recognized that atropisomers of the present compounds may exit. The present invention thus encompasses atropisomeric forms of compounds of formula I and II, as defined above, and in classes and sublcasses described above and herein. For definitions and an extensive discourse on atropisomers, see: Eliel, E. L. Stereochemistry of Organic Compounds (John Wiley & Sons, 1994, p 1142), which is incorporated herein by reference in its entirety.

The term “pharmaceutically acceptable salts” or “pharmaceutically acceptable salt” refers to salts derived from treating a compound of formula I with an organic or inorganic acid such as, for example, acetic, lactic, citric, cinnamic, tartaric, succinic, fumaric, maleic, malonic, mandelic, malic, oxalic, propionic, hydrochloric, hydrobromic, phosphoric, nitric, sulfuric, glycolic, pyruvic, methanesulfonic, ethanesulfonic, toluenesulfonic, salicylic, benzoic, or similarly known acceptable acids. In certain embodiments, the present invention provides the hydrochloride salt of a compound of formula I.

In some embodiments, certain reactions of the present invention are stereoselective. In other embodiments, certain reactions of the present invention are stereospecific.

The term “stereospecific” as used herein, is meant a reaction where starting materials differing only in their spacial configuration are converted to stereoisomerically distinct products. For example, in a stereospecific reaction, if the starting material is enantiopure (100% enantiomer excess “ee”), the final product will also be enantiopure. Similarly if the starting material has an enantiomer excess of about 50%, the final product will also have about a 50% enantiomer excess.

By “stereoselective” as used herein, it is meant a reaction where one stereoisomer is preferentially formed over another. Preferably, the process of the present invention will produce a dihydrobenzofuran having an enantiomer excess of at least about 30%, more preferably at least about 40%, and most preferably at least about 50%, where enantiomer excess is the mole percent excess of a single enantiomer over the racemate.

“Enantiomer excess” or “% ee” as used herein refers to the mole percent excess of a single enantiomer over the racemate.

As used herein, the term “chiral non-racemic” is used interchangeably with “enantiomerically enriched” and signifies that one enantiomer makes up more than 50% of the preparation. In certain embodiments, the term enantiomerically enriched signifies that at least 60% of the preparation is one of the enantiomers. In other embodiments, the term signifies that at least 75% of the preparation is one of the enantiomers. In other embodiments, the term signifies that at least 95% of the preparation is one of the enantiomers. is meant a nonracemic mixture of chiral molecules. In some embodiments, the chiral non-racemic compounds have more than about 30% ee. In other embodiments, the compounds have more than about 50% ee, or more than about 80% ee, or more than about 90% ee, or more than 95% ee, or more than 99% ee. The “*” in any chemical formula depicted herein indicates a chiral carbon.

The process of the present invention preferably produces dihydrobenzofuran derivatives having an enantiomer excess of at least about 30%, more preferably at least about 50%, and most preferably at least about 95%.

“Organic impurities” as used herein, refers to any organic by-product or residual material present in the desired dihydrobenzofuran product, and do not include residual solvents or water. “Total organic impurities” refer to the total amount of organic impurities present in the desired dihydrobenzofuran product. Percent organic impurities such as total organic impurities and single largest impurity, unless otherwise stated are expressed herein as HPLC area percent relative to the total area of the HPLC chromatogram. The HPLC area percent is reported at a wavelength where the desired product and maximum number of organic impurities absorb.

According to one aspect, the present invention provides a method for preparing an enantiomerically enriched compound of formula II: 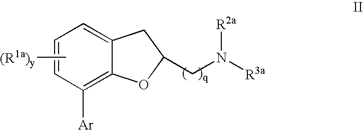
or a pharmaceutically acceptable salt thereof, wherein:
q is one or two;
each of R^{2a} and R^{3a} is independently hydrogen, methyl, ethyl, 2-fluoroethyl, 2,2-difluoroethyl or cyclopropyl;
each R^{1a} is independently hydrogen, halogen, OH, lower alkyl, lower alkoxy, lower haloalkyl, lower haloalkoxy, or CN;
Ar is thienyl, furyl, pyridyl, or phenyl, wherein Ar is optionally substituted with one or more R^{x} subsituents;
each R^{x} is independently selected from halogen, OH, lower alkyl, lower alkoxy, lower haloalkyl, lower haloalkoxy, or CN; and
y is 0, 1, 2, or 3.

As defined generally above, the Ar group of formula II is thienyl, furyl, pyridyl, or phenyl, wherein Ar is optionally substituted with one or more subsituents independently selected from halogen, OH, lower alkyl, lower alkoxy, haloalkyl, haloalkoxy, or CN. In certain embodiments, the Ar group of formula II is unsubstituted phenyl. In other embodiments, the Ar group of formula II is phenyl with at least one substituent in the ortho position. In other embodiments, the Ar group of formula II is phenyl with at least one substituent in the ortho position selected from halogen, lower alkyl, lower alkoxy, or trifluoromethyl. According to another aspect the present invention provides a compound of formula II wherein Ar is phenyl di-substituted in the ortho and meta positions with independently selected halogen, lower alkyl, or lower alkoxy. Yet another aspect of the present invention provides a compound of formula II wherein Ar is phenyl di-subsituted in the ortho and para positions with independently selected halogen, lower alkyl, or lower alkoxy. In other embodiment, the present invention provides a compound of formula II wherein Ar is phenyl di-subsituted in the two ortho positions with independently selected halogen, lower alkyl, or lower alkoxy. Exemplary substituents on the phenyl moiety of the Ar group of formula II include OMe, fluoro, chloro, methyl, and trifluoromethyl.

In certain embodiments, the present invention provides methods for preparing a compound of formula IIIa or IIIb: 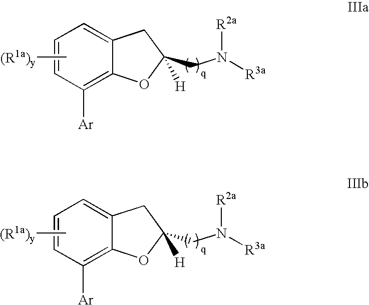
or a pharmaceutically acceptable salt thereof, wherein each R^{1a}, R^{2a}, R^{3a}, R^{x}, y, and q are as defined above for compounds of formula II and in classes and subclasses as described above and herein.

According to another embodiment, the present invention provides methods for preparing a compound of formula IIIc or IIId: 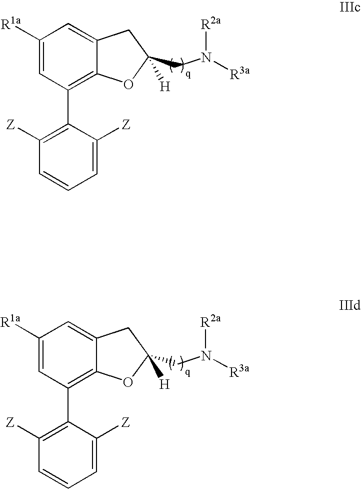
or a pharmaceutically acceptable salt thereof, wherein each of R^{1a}, R^{2a}, R^{3a}, Z, y, and q is as defined above for compounds of formula II and in classes and subclasses as described above and herein.

The invention also concerns intermediates of the processes of the present invention.

In certain embodiments, the present invention provides a method for preparing a compound of formula I: 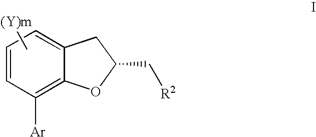
wherein:
m is 0-3;
R² is CN, N₃, or N(R³)(R⁴);
R³ and R⁴ are each independently hydrogen, an amine protecting group, C₁₋₆ alkyl, lower haloalkyl, 3-6 membered cycloaliphatic, or alkylcycloaliphatic, or R³ and R⁴ are taken together with the nitrogen to which they are attached to form a cyclic amine protecting group or a 3-6 membered saturated or partially unsaturated ring having 1-2 heteroatoms independently selected from nitrogen, oxygen, or sulfur;
each Y is independently hydrogen, chlorine, fluorine, CN, —OH, C₁₋₈ alkyl, C₁₋₆ perfluoroalkyl, C₁₋₆ alkoxy, C₁₋₆ perfluoroalkoxy, 6-10 membered aryl, 6-10 membered aryloxy, 5-10 membered heteroaryl having 1 to 4 heteroatoms independently selected from nitrogen, oxygen or sulfur, C₂₋₈ alkenyl, C₁₋₆ alkanesulfonamido, dialkylamino of 1 to 6 carbon atoms per alkyl moiety, C₃₋₈ cycloaliphatic, or 3-8 membered heterocycloalkyl having 1 to 3 heteroatoms independently selected from nitrogen, oxygen or sulfur; and
Ar is an optionally substituted 6-10 membered aryl or 5-10 membered heteroaryl having 1-4 heteroatoms independently selected from nitrogen, oxygen, or sulfur.
   wherein said method comprises one or more of the steps depicted in Scheme I above. In certain embodiments, said method comprises all of the steps depicted in Scheme I above.

It will be appreciated by one of ordinary skill in the art that, as used herein, the term “cyclic amino protecting group” includes, for example, phthalimide and derivatives thereof.

In some embodiments the invention provides a method for preparing a compound of formula I-a: 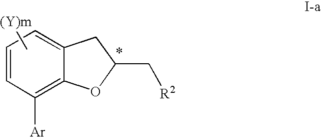
wherein:
m is 0-3;
R² is CN, N₃, or N(R³)(R⁴);
R³ and R⁴ are each independently hydrogen, an amine protecting group, C₁₋₆ alkyl, lower haloalkyl, 3-6 membered cycloaliphatic, or alkylcycloaliphatic, or R³ and R⁴ are taken together with the nitrogen to which they are attached to form a cyclic amine protecting group or a 3-6 membered saturated or partially unsaturated ring having 1-2 heteroatoms independently selected from nitrogen, oxygen, or sulfur;
each Y is independently hydrogen, chlorine, fluorine, CN, —OH, C₁₋₈ alkyl, C₁₋₆ perfluoroalkyl, C₁₋₆ alkoxy, C₁₋₆ perfluoroalkoxy, 6-10 membered aryl, 6-10 membered aryloxy, 5-10 membered heteroaryl having 1 to 4 heteroatoms independently selected from nitrogen, oxygen or sulfur, C₂₋₈ alkenyl, C₁₋₆ alkanesulfonamido, dialkylamino of 1 to 6 carbon atoms per alkyl moiety, C₃₋₈ cycloaliphatic, or 3-8 membered heterocycloalkyl having 1 to 3 heteroatoms independently selected from nitrogen, oxygen or sulfur; and
Ar is an optionally substituted 6-10 membered aryl or 5-10 membered heteroaryl having 1-4 heteroatoms independently selected from nitrogen, oxygen, or sulfur,
   comprising the steps of:
   providing a compound of the formula F-1: 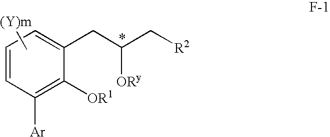
wherein:
m is 0-3;
R¹ is hydrogen or a suitable hydroxyl protecting group;
OR^{y} is a suitable leaving group;
R² is CN, N₃, or N(R³)(R⁴);
R³ and R⁴ are each independently hydrogen, an amine protecting group, C₁₋₆ alkyl, lower haloalkyl, 3-6 membered cycloaliphatic, or alkylcycloaliphatic, or R³ and R⁴ are taken together with the nitrogen to which they are attached to form a cyclic amine protecting group or a 3-6 membered saturated or partially unsaturated ring having 1-2 heteroatoms independently selected from nitrogen, oxygen, or sulfur;
each Y is independently hydrogen, chlorine, fluorine, CN, —OH, C₁₋₈ alkyl, C₁₋₆ perfluoroalkyl, C₁₋₆ alkoxy, C₁₋₆ perfluoroalkoxy, 6-10 membered aryl, 6-10 membered aryloxy, 5-10 membered heteroaryl having 1 to 4 heteroatoms independently selected from nitrogen, oxygen or sulfur, C₂-₈ alkenyl, C₁₋₆ alkanesulfonamido, dialkylamino of 1 to 6 carbon atoms per alkyl moiety, C₃₋₈ cycloaliphatic, or 3-8 membered heterocycloalkyl having 1 to 3 heteroatoms independently selected from nitrogen, oxygen or sulfur; and
Ar is an optionally substituted 6-10 membered aryl or 5-10 membered heteroaryl having 1-4 heteroatoms independently selected from nitrogen, oxygen, or sulfur, and
   converting the compound of formula F-1 to a compound of formula I-a or a pharmaceutically acceptable salt thereof.

It will be appreciated by one of ordinary skill in the art that when one of, or both of, R³ and R⁴ is hydrogen then nitrogen of R² is preferably protected prior to conversion of the hydroxyl group to a group of formula —OR^{y}.

In some embodiments, the Ar group of either of formulae I or I-a is: 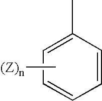
wherein:
n is 0-5; and
each Z is independently Cl, F, CN, —OH, cyano, C₁₋₆ alkyl, C₁₋₆ perfluoroalkyl, C₁₋₆ alkoxy, or C₁₋₆ perfluoroalkoxy.

In certain embodiments, at least one Z substituent is present on the phenyl ring at the ortho position. In other embodiments, one Z substituent is present on the phenyl ring at the ortho position and at least one other Z substituent is present on the phenyl ring at the ortho, meta, or para position.

According to one aspect the present invention provides a compound of either of formulae I or I-a wherein Ar is phenyl di-substituted in the ortho and meta positions with halogen, lower alkyl, or lower alkoxy. Yet another aspect of the present invention provides a compound of either of formulae I or I-a wherein Ar is phenyl di-subtsituted in the ortho and para positions with halogen, lower alkyl, or lower alkoxy. Yet another aspect of the present invention provides a compound of either of formulae I or I-a wherein Ar is phenyl di-substituted in both ortho positions with halogen, lower alkyl, or lower alkoxy. Exemplary substituents on the phenyl moiety of the Ar group of either of formulae I or I-a include OMe, fluoro, chloro, methyl, and trifluoromethyl.

In certain embodiments, the Ar group of either of formulae I or I-a is selected from the following:

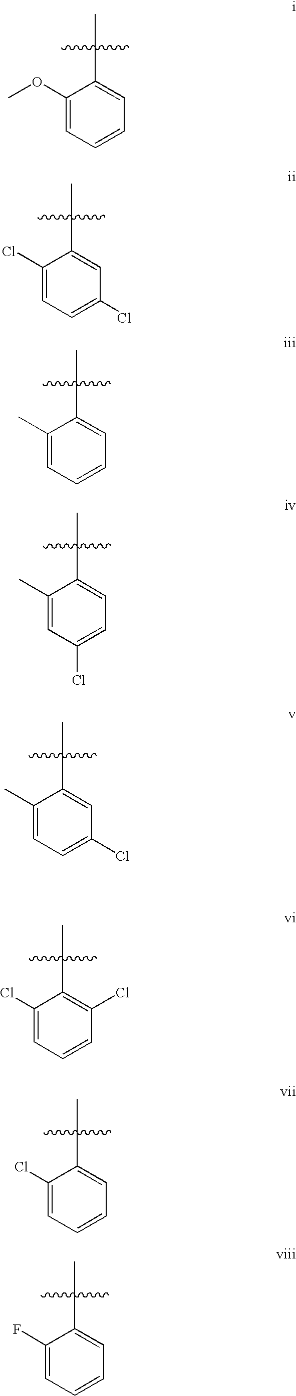

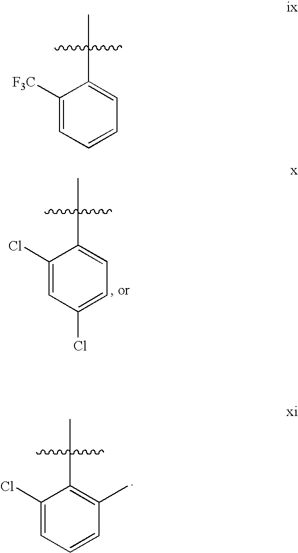

In certain embodiments, the present invention provides a method for preparing a compound of formula E-1: 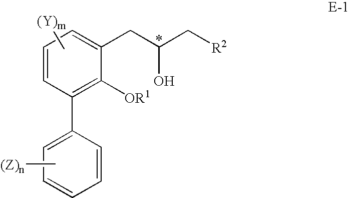
wherein:
m is 0-3;
n is 0-5;
R¹ is hydrogen or a suitable hydroxyl protecting group;
R² is CN, N₃, or N(R³)(R⁴);
R³ and R⁴ are each independently hydrogen, an amine protecting group, C₁₋₆ alkyl, lower haloalkyl, 3-6 membered cycloaliphatic, or alkylcycloaliphatic, or R³ and R⁴ are taken together with the nitrogen to which they are attached to form a cyclic amine protecting group or a 3-6 membered saturated or partially unsaturated ring having 1-2 heteroatoms independently selected from nitrogen, oxygen, or sulfur;
each Y is independently hydrogen, chlorine, fluorine, CN, —OH, C₁₋₈ alkyl, C₁₋₆ perfluoroalkyl, C₁₋₆ alkoxy, C₁₋₆ perfluoroalkoxy, 6-10 membered aryl, 6-10 membered aryloxy, 5-10 membered heteroaryl having 1 to 4 heteroatoms independently selected from nitrogen, oxygen or sulfur, C₂₋₈ alkenyl, C₁₋₆ alkanesulfonamido, dialkylamino of 1 to 6 carbon atoms per alkyl moiety, C₃₋₈ cycloaliphatic, or 3-8 membered heterocycloalkyl having 1 to 3 heteroatoms independently selected from nitrogen, oxygen or sulfur; and
each Z is independently Cl, F, CN, —OH, cyano, C₁₋₆ alkyl, C₁₋₆ perfluoroalkyl, C₁₋₆ alkoxy, or C₁₋₆ perfluoroalkoxy,
   comprising the steps of:
(a) providing a compound of formula C-1: 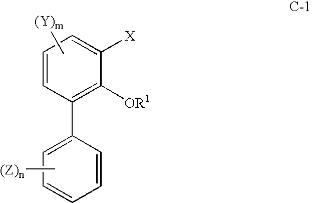
   wherein:
   m is 0-3;
   n is 0-5;
   X is halogen;
   R¹ is a suitable hydroxyl protecting group;
   each Y is independently hydrogen, chlorine, fluorine, CN, —OH, C₁₋₈ alkyl, C₁₋₆ perfluoroalkyl, C₁₋₆ alkoxy, C₁₋₆ perfluoroalkoxy, 6-10 membered aryl, 6-10 membered aryloxy, 5-10 membered heteroaryl having 1 to 4 heteroatoms independently selected from nitrogen, oxygen or sulfur, C₂₋₈ alkenyl, C₁₋₆ alkanesulfonamido, dialkylamino of 1 to 6 carbon atoms per alkyl moiety, C₃₋₈ cycloaliphatic, or 3-8 membered heterocycloalkyl having 1 to 3 heteroatoms independently selected from nitrogen, oxygen or sulfur; and
   each Z is independently Cl, F, CN, —OH, cyano, C₁₋₆ alkyl, C₁₋₆ perfluoroalkyl, C₁₋₆ alkoxy, or C₁₋₆ perfluoroalkoxy,
(b) converting the compound of formula C-1 to a compound of formula C-2: 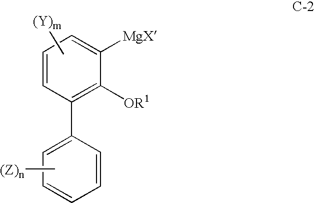
   wherein:
   m is 0-3;
   n is 0-5;
   X′ is halogen;
   R′ is a suitable hydroxyl protecting group;
   each Y is independently hydrogen, chlorine, fluorine, CN, —OH, C₁₋₈ alkyl, C₁₋₆ perfluoroalkyl, C₁₋₆ alkoxy, C₁₋₆ perfluoroalkoxy, 6-10 membered aryl, 6-10 membered aryloxy, 5-10 membered heteroaryl having 1 to 4 heteroatoms independently selected from nitrogen, oxygen or sulfur, C₂₋₈ alkenyl, C₁₋₆ alkanesulfonamido, dialkylamino of 1 to 6 carbon atoms per alkyl moiety, C₃₋₈ cycloaliphatic, or 3-8 membered heterocycloalkyl having 1 to 3 heteroatoms independently selected from nitrogen, oxygen or sulfur; and
   each Z is independently Cl, F, CN, —OH, cyano, C₁₋₆ alkyl, C₁₋₆ perfluoroalkyl, C₁₋₆ alkoxy, or C₁₋₆ perfluoroalkoxy,
(c) reacting the compound of formula C-2 with a chiral non-racemic epoxide of the formula: 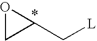
   wherein L is a leaving group, to produce a compound of formula D-1: 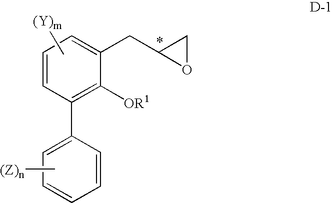
   wherein:
   m is 0-3;
   n is 0-5;
   R¹ is a suitable hydroxyl protecting group;
   each Y is independently hydrogen, chlorine, fluorine, CN, —OH, C₁₋₈ alkyl, C₁₋₆ perfluoroalkyl, C₁₋₆ alkoxy, C₁₋₆ perfluoroalkoxy, 6-10 membered aryl, 6-10 membered aryloxy, 5-10 membered heteroaryl having 1 to 4 heteroatoms independently selected from nitrogen, oxygen or sulfur, C₂₋₈ alkenyl, C₁₋₆ alkanesulfonamido, dialkylamino of 1 to 6 carbon atoms per alkyl moiety, C₃₋₈ cycloaliphatic, or 3-8 membered heterocycloalkyl having 1 to 3 heteroatoms independently selected from nitrogen, oxygen or sulfur; and
   each Z is independently Cl, F, CN, —OH, cyano, C₁₋₆ alkyl, C₁₋₆ perfluoroalkyl, C₁₋₆ alkoxy, or C₁₋₆ perfluoroalkoxy,
(d) and converting the compound of formula D-1 to a compound of formula E-1.

In certain embodiments, the compound of formula D-1 is converted to a compound of formula E-1 by treating the compound of formula D-1 with a compound of formula H—R² and/or M-R² optionally in the presence of a suitable solvent. In certain embodiments, R is —N(R³)(R⁴) and is phthalimide. In other embodiments, R² is N₃.

In certain other embodiments, the preparation of a compound of formula E-1 comprises:
(a) providing a compound of the formula: 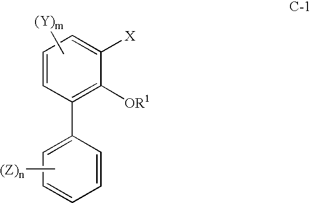
   wherein:
   m is 0-3;
   n is 0-5;
   X is halogen;
   R¹ is a suitable hydroxyl protecting group;
   each Y is independently hydrogen, chlorine, fluorine, CN, —OH, C₁₋₈ alkyl, C₁₋₆ perfluoroalkyl, C₁₋₆ alkoxy, C₁₋₆ perfluoroalkoxy, 6-10 membered aryl, 6-10 membered aryloxy, 5-10 membered heteroaryl having 1 to 4 heteroatoms independently selected from nitrogen, oxygen or sulfur, C₂₋₈ alkenyl, C₁₋₆ alkanesulfonamido, dialkylamino of 1 to 6 carbon atoms per alkyl moiety, C₃₋₈ cycloaliphatic, or 3-8 membered heterocycloalkyl having 1 to 3 heteroatoms independently selected from nitrogen, oxygen or sulfur; and
   each Z is independently Cl, F, CN, —OH, cyano, C₁₋₆ alkyl, C₁₋₆ perfluoroalkyl, C₁₋₆ alkoxy, or C₁₋₆ perfluoroalkoxy,
(b) converting the compound of formula C-1 to a compound of formula C-2: 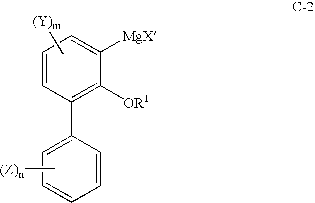
   wherein:
   m is 0-3;
   n is 0-5;
   X′ is halogen;
   R¹ is a suitable hydroxyl protecting group;
   each Y is independently hydrogen, chlorine, fluorine, CN, —OH, C₁₋₈ alkyl, C₁₋₆ perfluoroalkyl, C₁₋₆ alkoxy, C₁₋₆ perfluoroalkoxy, 6-10 membered aryl, 6-10 membered aryloxy, 5-10 membered heteroaryl having 1 to 4 heteroatoms independently selected from nitrogen, oxygen or sulfur, C₂₋₈ alkenyl, C₁₋₆ alkanesulfonamido, dialkylamino of 1 to 6 carbon atoms per alkyl moiety, C₃₋₈ cycloaliphatic, or 3-8 membered heterocycloalkyl having 1 to 3 heteroatoms independently selected from nitrogen, oxygen or sulfur; and
   each Z is independently Cl, F, CN, —OH, cyano, C₁₋₆ alkyl, C₁₋₆ perfluoroalkyl, C₁₋₆ alkoxy, or C₁₋₆ perfluoroalkoxy,
(c) reacting the compound of formula C-2 with a chiral non-racemic epoxide of formula 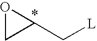
   wherein L is a leaving group, to produce at least one compound of formula: 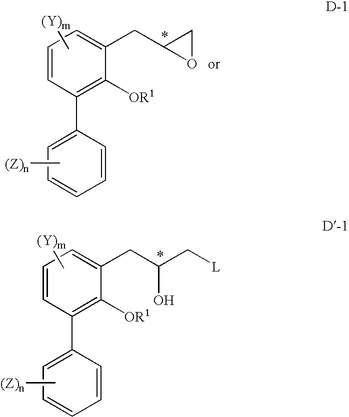
(d) converting the compound of formula D-1 or D′-1 to a compound of formula E-1.

In certain embodiments, the compound of formula D-1 or D′-1 is converted to a compound of formula E-1 by treating the compound of formula D-1 or D′-1 with a compound of formula H—R² and/or M-R² optionally in the presence of a suitable solvent. In certain embodiments, R² is —N(R³)(R⁴) and is phthalimide.

In certain aspects, the invention concerns a process where the preparation of the compound of formula I comprises:
(a) providing a compound of the formula: 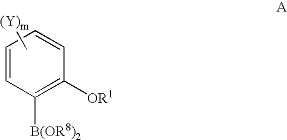
   wherein:
   m is 0-3;
   R¹ is hydrogen or a suitable hydroxyl protecting group;
   each Y is independently hydrogen, chlorine, fluorine, CN, —OH, C₁₋₈ alkyl, C₁₋₆ perfluoroalkyl, C₁₋₆ alkoxy, C₁₋₆ perfluoroalkoxy, 6-10 membered aryl, 6-10 membered aryloxy, 5-10 membered heteroaryl having 1 to 4 heteroatoms independently selected from nitrogen, oxygen or sulfur, C₂₋₈ alkenyl, C₁₋₆ alkanesulfonamido, dialkylamino of 1 to 6 carbon atoms per alkyl moiety, C₃₋₈ cycloaliphatic, or 3-8 membered heterocycloalkyl having 1 to 3 heteroatoms independently selected from nitrogen, oxygen or sulfur; and
   each R⁸ is independently hydrogen or C₁₋₆ alkyl,
(b) contacting the compound of formula A with a compound of formula A-1 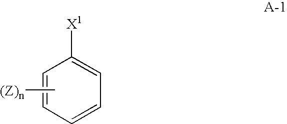
   wherein:
   n is 0-5;
   each Z is independently Cl, F, CN, —OH, cyano, C₁₋₆ alkyl, C₁₋₆ perfluoroalkyl, C₁₋₆ alkoxy, or C₁₋₆ perfluoroalkoxy; and
   X¹ is Cl, Br, I, triflate (—OSO₂CF₃) or other perfluoroalkylsulfonate,
      in the presence of a palladium catalyst to produce a compound of the formula B-1: 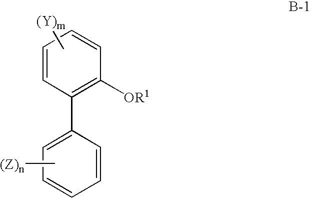
   wherein:
   m is 0-3;
   n is 0-5;
   R¹ is hydrogen or a suitable hydroxyl protecting group;
   each Y is independently hydrogen, chlorine, fluorine, CN, —OH, C₁₋₈ alkyl, C₁₋₆ perfluoroalkyl, C₁₋₆ alkoxy, C₁₋₆ perfluoroalkoxy, 6-10 membered aryl, 6-10 membered aryloxy, 5-10 membered heteroaryl having 1 to 4 heteroatoms independently selected from nitrogen, oxygen or sulfur, C₂₋₈ alkenyl, C₁₋₆ alkanesulfonamido, dialkylamino of 1 to 6 carbon atoms per alkyl moiety, C₃₋₈ cycloaliphatic, or 3-8 membered heterocycloalkyl having 1 to 3 heteroatoms independently selected from nitrogen, oxygen or sulfur; and
   each Z is independently Cl, F, CN, —OH, cyano, C₁₋₆ alkyl, C₁₋₆ perfluoroalkyl, C₁₋₆ alkoxy, or C₁₋₆ perfluoroalkoxy,
      and
(c) contacting the compound of formula B-1 with a halogenating agent to produce a compound of formula C-1: 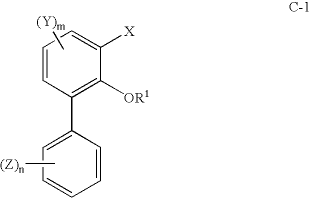
   wherein:
   m is 0-3;
   n is 0-5;
   X is halogen;
   R¹ is a suitable hydroxyl protecting group;
   each Y is independently hydrogen, chlorine, fluorine, CN, —OH, C₁₋₈ alkyl, C₁₋₆ perfluoroalkyl, C₁₋₆ alkoxy, C₁₋₆ perfluoroalkoxy, 6-10 membered aryl, 6-10 membered aryloxy, 5-10 membered heteroaryl having 1 to 4 heteroatoms independently selected from nitrogen, oxygen or sulfur, C₂₋₈ alkenyl, C₁₋₆ alkanesulfonamido, dialkylamino of 1 to 6 carbon atoms per alkyl moiety, C₃₋₈ cycloaliphatic, or 3-8 membered heterocycloalkyl having 1 to 3 heteroatoms independently selected from nitrogen, oxygen or sulfur; and
   each Z is independently Cl, F, CN, —OH, cyano, C₁₋₆ alkyl, C₁₋₆ perfluoroalkyl, C₁₋₆ alkoxy, or C₁₋₆ perfluoroalkoxy.

In certain embodiments, R¹ is C₁₋₄ alkyl. In some embodiments, X¹ is Br. In certain embodiments, step (b) above (the Suzuki coupling step) is performed using conditions that are well known in the art. See, for example, Miyaura, N.; Suzuki, A. Chem. Rev. 1995, 95, 2457. In certain embodiments, the Suzuki coupling at step (b) is performed in the presence of a palladium containing compound. In other embodiments, the palladium containing compound is Pd(PPh₃)₄. The reaction may also be performed in the presence of a base. In certain embodiments, the base is MOH, M₂CO₃, or M₃PO₄, wherein each M is independently an alkali metal. In some embodiments, the alkali metal is sodium, potassium, or lithium. In other embodiments, step (b) is performed in the presence of NaOH. The reaction is optionally carried out in the presence of a solvent. Suitable solvents for the coupling at step (b) include dimethoxyethane, toluene, ethanol, isopropanol, methanol, and tetrahydrofuran (THF). In certain embodiments, the suitable solvent for the coupling step of (b) includes water. In certain embodiments, the Suzuki coupling reaction is carried out at a temperature in the range of about 20° C. to about the temperature sufficient to reflux the solvent or mixture of thereof. In other embodiments, the coupling reaction is carried out at about 60 to about 90° C. In still other embodiments, the coupling reaction is carried out at about 70 to about 80° C.

Some embodiments of the invention concern a process where the conversion of the compound of formula C-1 to the compound of formula D-1 comprises:
(a) contacting the compound of formula C-1 with a Grignard reagent or magnesium metal to form a compound of formula C-2: 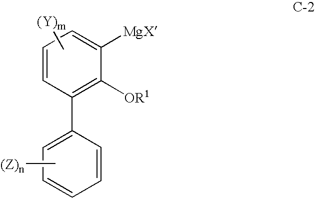
   wherein:
   m is 0-3;
   n is 0-5;
   X′ is halogen;
   R¹ is a suitable hydroxyl protecting group;
   each Y is independently hydrogen, chlorine, fluorine, CN, —OH, C₁₋₈ alkyl, C₁₋₆ perfluoroalkyl, C₁₋₆ alkoxy, C₁₋₆ perfluoroalkoxy, 6-10 membered aryl, 6-10 membered aryloxy, 5-10 membered heteroaryl having 1 to 4 heteroatoms independently selected from nitrogen, oxygen or sulfur, C₂₋₈ alkenyl, C₁₋₆ alkanesulfonamido, dialkylamino of 1 to 6 carbon atoms per alkyl moiety, C₃₋₈ cycloaliphatic, or 3-8 membered heterocycloalkyl having 1 to 3 heteroatoms independently selected from nitrogen, oxygen or sulfur; and
   each Z is independently Cl, F, CN, —OH, cyano, C₁₋₆ alkyl, C₁₋₆ perfluoroalkyl, C₁₋₆ alkoxy, or C₁₋₆ perfluoroalkoxy,
(b) contacting the compound of formula C-2 with a chiral non-racemic epoxide of formula 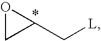
   wherein L is a leaving group, to form a compound of the formula D′-1: 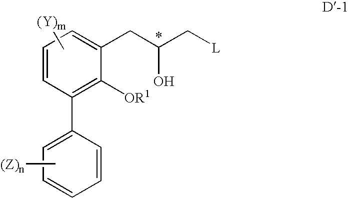
(c) and contacting the compound of formula D′-1 with a suitable base to produce a compound of formula D-1:

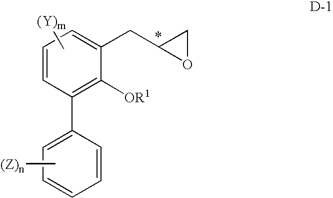

In certain embodiments, the step of contacting a compound of formula C-2 and 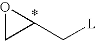
occurs in the presence of a copper salt. In some embodiments, this step occurs at a temperature of about −15° C. to about −35° C. In yet other embodiments, this step occurs at a temperature of about −20° C. to about −25° C. In some embodiments, the copper salt is CuCN, Li₂CuCl₄ or CuI. According to one embodiment, the copper salt is CuI. In certain embodiments, the Grignard reagent is isopropyl magnesium chloride.

Some embodiments of the invention concern a process where the compound of formula E is: 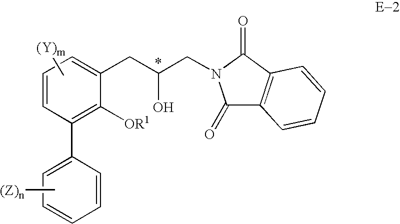
wherein:
m is 0-3;
n is 0-5;
R¹ is hydrogen or a suitable hydroxyl protecting group;
each Y is independently hydrogen, chlorine, fluorine, CN, —OH, C₁₋₈ alkyl, C₁₋₆ perfluoroalkyl, C₁₋₆ alkoxy, C₁₋₆ perfluoroalkoxy, 6-10 membered aryl, 6-10 membered aryloxy, 5-10 membered heteroaryl having 1 to 4 heteroatoms independently selected from nitrogen, oxygen or sulfur, C₂₋₈ alkenyl, C₁₋₆ alkanesulfonamido, dialkylamino of 1 to 6 carbon atoms per alkyl moiety, C₃₋₈ cycloaliphatic, or 3-8 membered heterocycloalkyl having 1 to 3 heteroatoms independently selected from nitrogen, oxygen or sulfur; and
each Z is independently Cl, F, CN, —OH, cyano, C₁₋₆ alkyl, C₁₋₆ perfluoroalkyl, C₁₋₆ alkoxy, or C₁₋₆ perfluoroalkoxy.

In certain of these processes the compound of formula E-2 is contacted with R^{y}Cl and a suitable base to produce a compound of formula F-1: 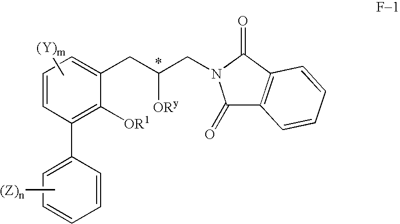
wherein R¹, n, m, Y, and Z are as defined above and —OR^{y} is optionally substituted alkylsulphonyloxy, optionally substituted alkenylsulfonyloxy, or optionally substituted arylsulfonyloxy. Examples of such R^{y} groups include methanesulfonyl (mesyl), tosyl, triflate, nitro-phenylsulfonyl (nosyl), and bromo-phenylsulfonyl (brosyl).

In certain embodiments, the compound of formula F-1, as defined herein, is contacted with a methyl ether cleaving agent to produce a compound of formula G-1: 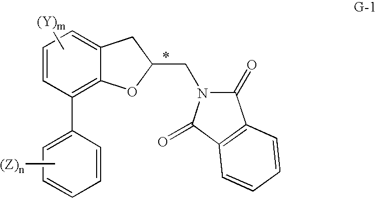
wherein n, m, Y, and Z are as defined herein.

In certain embodiments, the phthalimide moiety of compound G-1 is removed to form a compound of formula I-a: 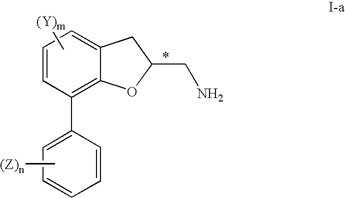
wherein n, m, Y, and Z are as defined herein. It will be appreciated by one of ordinary skill in the art that when other amino protecting groups are utilized, i.e. instead of the phthalimide group, then those protecting groups can be removed by suitable means to form a compound of formula I-a.

In some embodiments, the methyl ether cleaving agent is selected from BI₃, BBr₃, AlI₃, or AlBr₃. In other embodiments, said agent is BBr₃.

In certain embodiments, the phthalimide moiety of the compound of formula G-1 is converted to a compound of formula I-a in a process comprising contacting the compound of formula G-1 with a hydrazine. In some embodiments, the hydrazine is hydrazine hydrate.

In certain preferred embodiments, the hydrazine contacting step occurs in the presence of an alcohol or THF solvent or mixtures thereof.

In some aspects, the invention relates to processes where the compound of formula I is 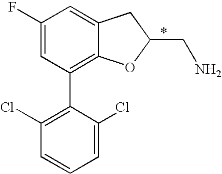
or a pharmaceutically acceptable salt thereof.

As described generally above, the present invention provides methods for preparing a compound of formula I: 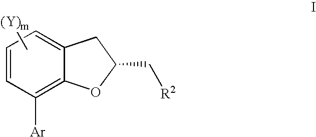
wherein:
m is 0-3;
R² is CN, N₃, or N(R³)(R⁴);
R³ and R⁴ are each independently hydrogen, an amine protecting group, C₁₋₆ alkyl, lower haloalkyl, 3-6 membered cycloaliphatic, or alkylcycloaliphatic, or R³ and R⁴ are taken together with the nitrogen to which they are attached to form a cyclic amine protecting group or a 3-6 membered saturated or partially unsaturated ring having 1-2 heteroatoms independently selected from nitrogen, oxygen, or sulfur;
each Y is independently hydrogen, chlorine, fluorine, CN, —OH, C₁₋₈ alkyl, C₁₋₆ perfluoroalkyl, C₁₋₆ alkoxy, C₁₋₆ perfluoroalkoxy, 6-10 membered aryl, 6-10 membered aryloxy, 5-10 membered heteroaryl having 1 to 4 heteroatoms independently selected from nitrogen, oxygen or sulfur, C₂₋₈ alkenyl, C₁₋₆ alkanesulfonamido, dialkylamino of 1 to 6 carbon atoms per alkyl moiety, C₃₋₈ cycloaliphatic, or 3-8 membered heterocycloalkyl having 1 to 3 heteroatoms independently selected from nitrogen, oxygen or sulfur; and
Ar is an optionally substituted 6-10 membered aryl or 5-10 membered heteroaryl having 1-4 heteroatoms independently selected from nitrogen, oxygen, or sulfur.

It will be appreciated that when the R² group of formula I is CN, the cyano group may be reduced to form a compound of formula I′: 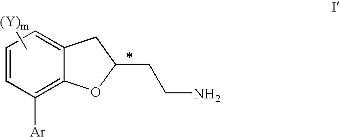
wherein m, Y, and Ar as defined herein.

Thus, another embodiment of the present invention provides a method for preparing a compound of formula I′: 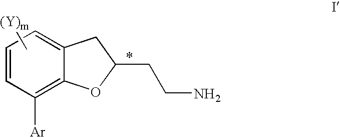
wherein:
m is 0-3;
each Y is independently hydrogen, chlorine, fluorine, CN, —OH, C₁₋₈ alkyl, C₁₋₆ perfluoroalkyl, C₁₋₆ alkoxy, C₁₋₆ perfluoroalkoxy, 6-10 membered aryl, 6-10 membered aryloxy, 5-10 membered heteroaryl having 1 to 4 heteroatoms independently selected from nitrogen, oxygen or sulfur, C₂₋₈ alkenyl, C₁₋₆ alkanesulfonamido, dialkylamino of 1 to 6 carbon atoms per alkyl moiety, C₃₋₈ cycloaliphatic, or 3-8 membered heterocycloalkyl having 1 to 3 heteroatoms independently selected from nitrogen, oxygen or sulfur; and
Ar is an optionally substituted 6-10 membered aryl or 5-10 membered heteroaryl having 1-4 heteroatoms independently selected from nitrogen, oxygen, or sulfur,
   comprising the steps of:
(a) providing a compound of formula D: 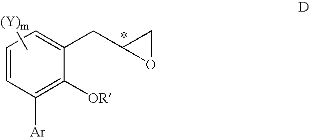
   wherein:
   m is 0-3;
   R¹ is a suitable hydroxyl protecting group;
   each Y is independently hydrogen, chlorine, fluorine, CN, —OH, C₁₋₈ alkyl, C₁₋₆ perfluoroalkyl, C₁₋₆ alkoxy, C₁₋₆ perfluoroalkoxy, 6-10 membered aryl, 6-10 membered aryloxy, 5-10 membered heteroaryl having 1 to 4 heteroatoms independently selected from nitrogen, oxygen or sulfur, C₂₋₈ alkenyl, C₁₋₆ alkanesulfonamido, dialkylamino of 1 to 6 carbon atoms per alkyl moiety, C₃₋₈ cycloaliphatic, or 3-8 membered heterocycloalkyl having 1 to 3 heteroatoms independently selected from nitrogen, oxygen or sulfur; and
   Ar is an optionally substituted 6-10 membered aryl or 5-10 membered heteroaryl having 1-4 heteroatoms independently selected from nitrogen, oxygen, or sulfur,
(b) treating said compound of formula D with a compound M-CN, wherein M is a suitable metal, to form a compound of formula H: 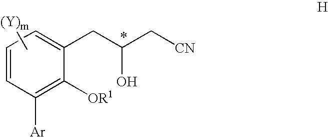
   wherein:
   m is 0-3;
   R¹ is a suitable hydroxyl protecting group;
   each Y is independently hydrogen, chlorine, fluorine, CN, —OH, C₁₋₈ alkyl, C₁₋₆ perfluoroalkyl, C₁₋₆ alkoxy, C₁₋₆ perfluoroalkoxy, 6-10 membered aryl, 6-10 membered aryloxy, 5-10 membered heteroaryl having 1 to 4 heteroatoms independently selected from nitrogen, oxygen or sulfur, C₂₋₈ alkenyl, C₁₋₆ alkanesulfonamido, dialkylamino of 1 to 6 carbon atoms per alkyl moiety, C₃₋₈ cycloaliphatic, or 3-8 membered heterocycloalkyl having 1 to 3 heteroatoms independently selected from nitrogen, oxygen or sulfur; and
   Ar is an optionally substituted 6-10 membered aryl or 5-10 membered heteroaryl having 1-4 heteroatoms independently selected from nitrogen, oxygen, or sulfur,
(c) converting the hydroxyl group of compound H to a suitable leaving group,
(d) cyclizing compound H to form a compound of formula J: 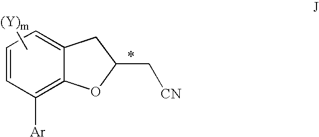
   wherein m, Ar, and Y are as defined herein; and
(e) reducing the cyano group to form a compound of formula I′.

In other aspects, the invention concerns the products, including intermediates and by-products, of the processes described herein.

In certain embodiments, compounds of the present invention are synthesized according to the following synthetic Scheme II below. One skilled in the art will recognize that this asymmetric synthesis could be used to synthesize the opposite enantiomer in enantiomeric excess using the appropriate glycidyl tosylate.

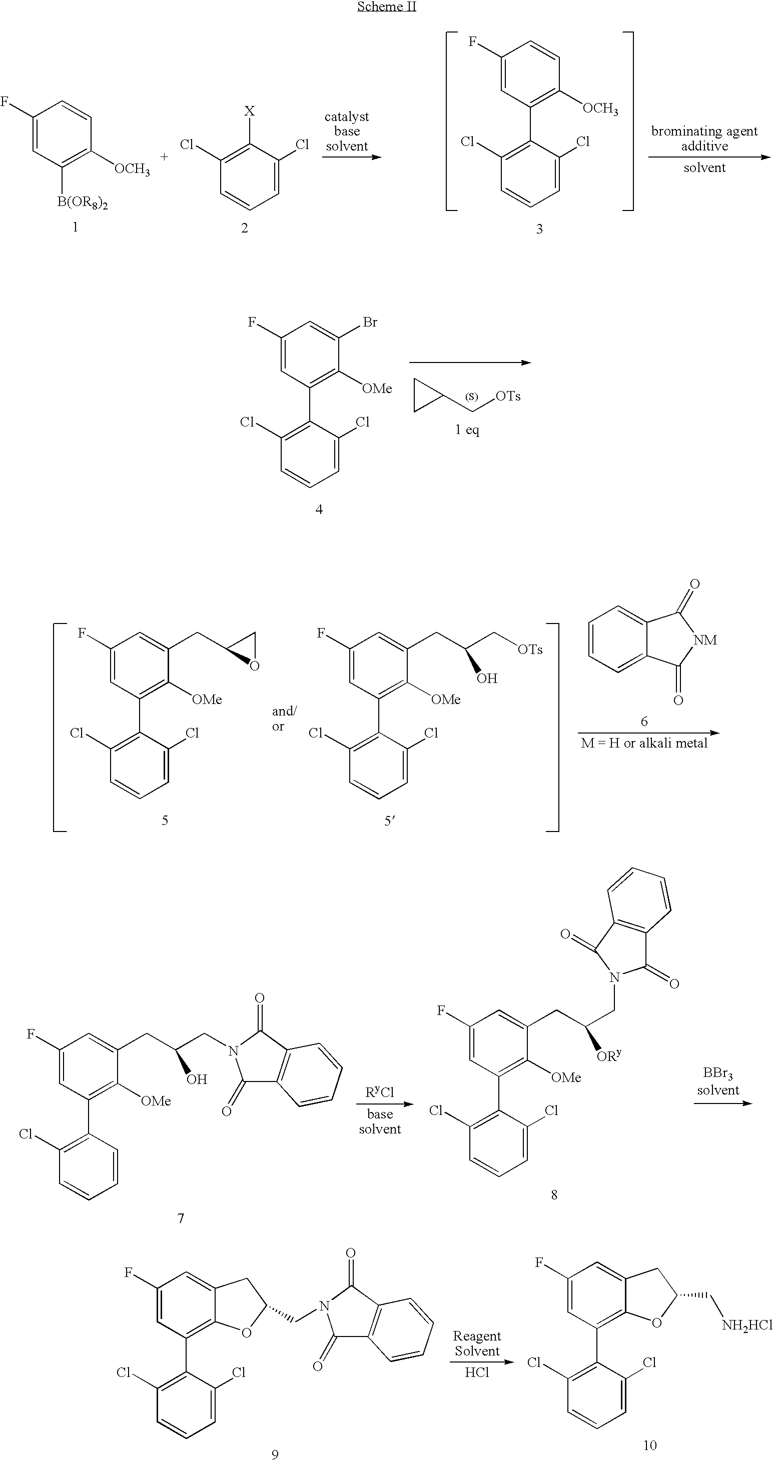

Each of the above reaction steps are described more generally below. One skilled in the art will recognize that these reaction steps described below and as illustrated in Scheme II can be generally applied to form compounds of the present invention.

Catalyst and reaction conditions for the Suzuki reaction of Scheme III below are well known in the art. See, for example, Miyaura, N.; Suzuki, A. Chem. Rev. 1995, 95, 2457. In certain embodiments, the reaction shown in Scheme III is performed in the presence of a palladium containing compound. In certain embodiments, the palladium containing compound is Pd(PPh₃)₄. The reaction may also be formed in the presence of a base. In certain embodiments, the base is MOH, M₂CO₃, or M₃PO₄, where M is an alkali metal. In some embodiments, the alkali metal is sodium, potassium, or lithium. The reaction may be carried out in the presence of a solvent. Preferred solvents include dimethoxyethane, toluene, ethanol, isopropanol, methanol, tetrahydrofuran (THF) and mixtures thereof. In other embodiments, the additionally comprises contains water. In certain embodiments, the reaction is carried out at a temperature in the range of about 40° C. to about the temperature for refluxing the solvent.

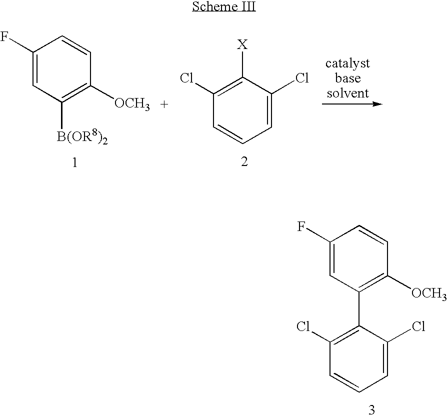

In certain preferred embodiments, the brominating agents used in Scheme IV below is bromine or any of the compounds containing N—Br group (e.g., N-bromosuccinimide). Other brominating agents are known to those skilled in the art. In certain embodiments, one or more additives are used in the reaction. These additives include inorganic acid such as H₂SO₄ (used with a N—Br brominating agent), Lewis acid, or AcONa (used with bromine). Preferred solvents for the reaction include dioxane, THF, acetic acid, or a chlorinated solvent. Chlorinated solvents include CH₂Cl₂, CHCl₃, CCl₄, dichloroethane, or others. In some embodiments, the reaction is preformed at a temperature of about 18° C. to about solvent reflux. In other embodiments, the additive is p-toluene sulfonic acid and the solvent is acetic acid or formic acid.

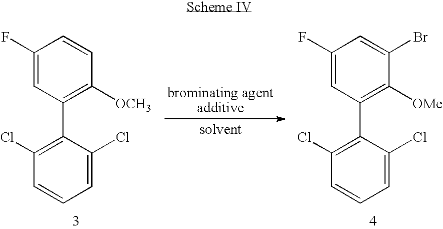

In some embodiments, the magnesium containing reagent used in Scheme V below is Mg metal or RMgX, where R is alkyl and X is halogen. Preferred solvents include THF and dialkyl ether. Preferred catalysts include copper salts. Such copper salts are preferably used in an amount of about 1 to 100 mol % relative to the starting material. In certain embodiments, the copper salt is CuI. Preferred bases used in this scheme include MOH and M₂CO₃, where each M is an alkali metal. “OTs” in Scheme V represents the leaving group tosylate. The base can be used neat or as an aqueous solution. In certain embodiments, the Grignard formation is carried out at a temperature of from about −50° C. to about −10° C. In other embodiments, the reaction is performed at below about −18° C. ln certain embodiments the alkylation step is performed at about 0° C. to about 50° C. In other embodiments, as more fully described in Scheme X, epoxide formation is not necessary and thus that step (as described below in Example 3) can be eliminated.

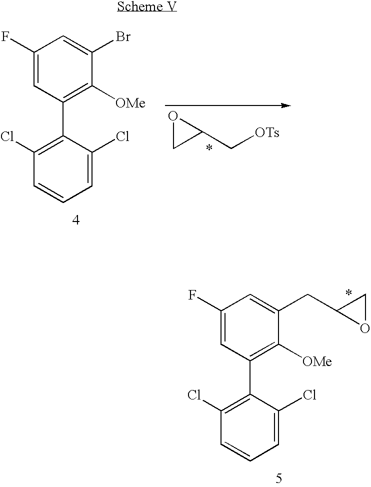

In the reaction shown in Scheme VI below, Pht-NH and Pht-NM represents phthalimide and its salt where M is a metal respectively. In some embodiments of Scheme VI, the phthalimide reagent does not need to be a mixture and may be for example only Pht-NM as more fully described in Scheme X. One skilled in the art will recognize that other reagents may be used that provide a protected amine or a nonreactive amine such as those having the formula HNR³R⁴ and/or MNR³R⁴ where R³ and R⁴ are defined as previously herein. In certain embodiment, M is a Group I metal. In some embodiments, M is K, Li, or Na. In some preferred embodiments, the amount of Pht-NH is greater or equal to 1 equivalent of Pht-NM. In certain embodiments, about 0.1 to about 1 equivalents of Pht-NM per equivalent of Pht-NH is preferred. Preferred solvents for this step include DMF, DMSO and NMP. In some embodiments, the reaction is performed at a temperature of from about 40° C. to about 130° C.

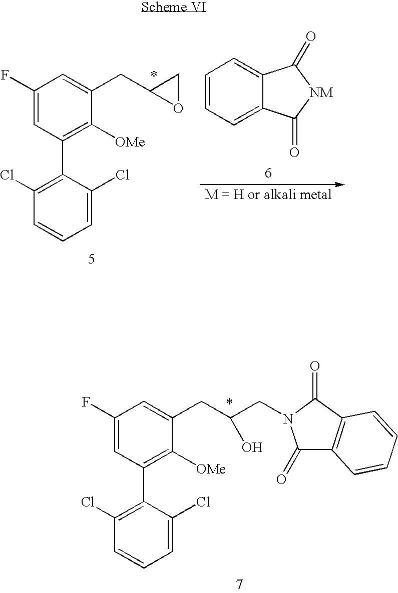

In Scheme VII below, the base is in certain embodiments any tertiary amine or M₂CO₃, where M is a Group I metal. Preferred solvents include toluene or any chlorinated solvent, THF, and dialkyl ether. In some embodiments, the preferred reaction temperature is from about 0° C. to about 60° C. In certain embodiments, OR^{y} is a mesylate (“OMs”) leaving group.

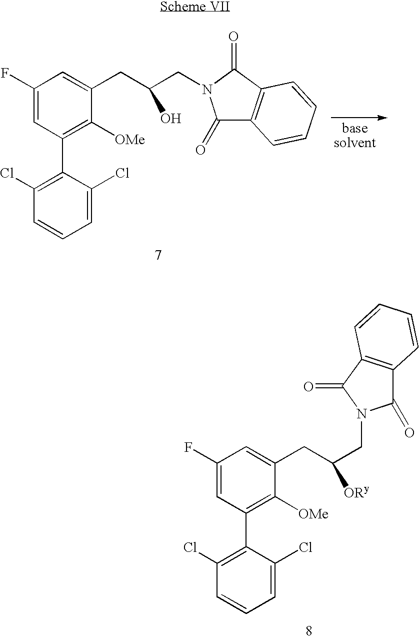

For the reaction shown in Scheme VIII below, a methyl ether cleaving agent such as BBr₃ is used. Other cleaving agents that may be used include those described in Greene, T. W., Wuts, P. G. M., Protective Groups in Organic Synthesis, 3rd Ed.; Wiley & Sons, New York, 1999. Preferred solvents include any chlorinated solvent or toluene. In some embodiments, preferred reaction temperatures are from about −10° C. to about 25° C.

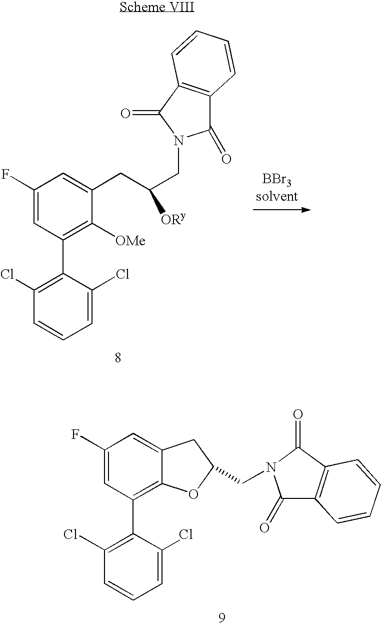

If a protected amine is used in the above reaction scheme (where R³ and/or R⁴ are amine protecting group(s)), preferably the compound formed after cyclization is deprotected. Scheme IX below, shows one such method for deprotection to form the compound of formula I where R³ and R⁴ are hydrogen. In certain preferred embodiments of Scheme IX, the phthalimide amine protecting group is removed using reagent R′NHNH₂ where R′ is H, C₁₋₆ alkyl, or aryl. Preferred solvents for the deprotection step include THF and R″OH, where R″ is C₁ to C₆ alkyl. Preferred temperatures for the deprotection step is from about 40° C. to about solvent reflux. Preferred solvents for the salt formation include dialkyl ether or R″OH where R″ is C₁ to C₆ alkyl. A pharmaceutically acceptable salt may optionally be formed of formula IV using any pharmaceutically acceptable acid such as HCl. Preferred temperatures for the salt formation include about 18° C. to about 30° C.

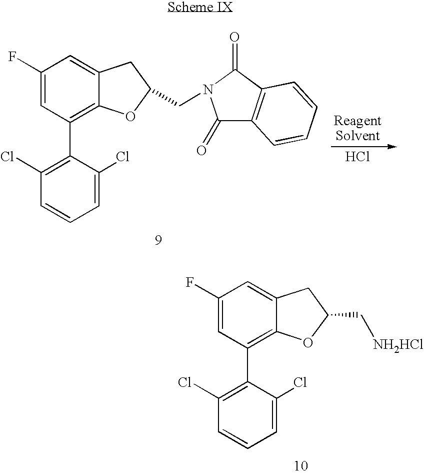

A method for making 2(R)-7-(2,6-dichlorophenyl)-5-fluoro-2,3-dihydro-2-aminomethylbenzofuran hydrochloride is shown in Scheme X below. One skilled in the art will recognize that this asymmetric synthesis could be used to synthesize the corresponding “S” enantiomer in enantiomeric excess using the appropriate glycidyl tosylate in Step 3a below. Each Step of Scheme X is discussed in more detail below.

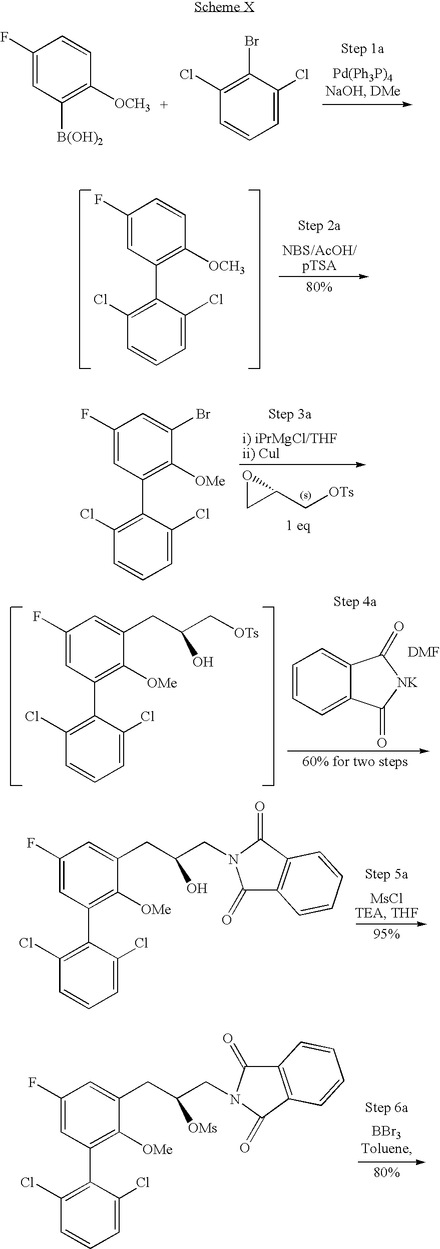

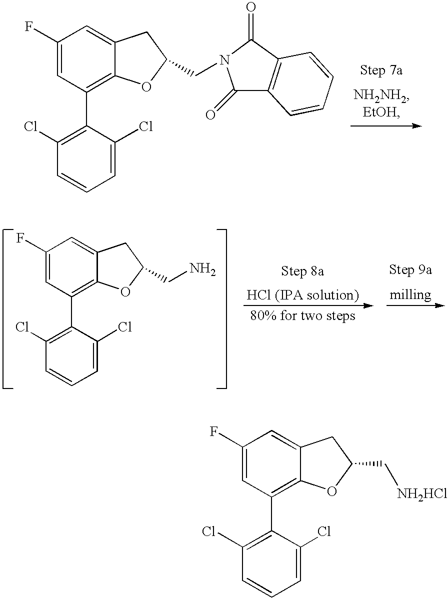

In Step 1a of Scheme X above, 2,6-dichlorobromobenzene and 2-methoxy-5-fluoro-benzeneboronic acid are reacted via a Suzuki coupling reaction to form 2′,6′-dichloro-5-fluoro-2-methoxy-biphenyl. The coupling is carried out in the presence of a palladium catalyst and a base. The coupling is preferably carried out in the presence of a solvent such as dimethoxyethane, ethanol, tetrahydrofuran, isopropanol, or methanol, or combinations thereof.

In an embodiment of the invention, an aqueous solution of sodium hydroxide is added to a mixture of 2,6-dichlorobromobenzene, 2-methoxy-5-fluorobenzeneboronic acid, tetrakis(triphenylphosphine)palladium(0) (Pd(PPh₃)₄) and dimethoxyethane (DME). The molar ratio of 2-methoxy-5-fluoro-benzeneboronic acid to dichlorobromobenzene, may be for example from about 1:1 to about 2:1. Preferably NaOH is used in an amount of from about 2 to about 5 equivalents based on the dichlorobromobenzene. The palladium catalyst may be used in an amount of from about 1% to 5% based on the dichlorobromobenzene. In another embodiment, other bases may be used in Step 1a, such as KOH in place of NaOH. For minimization of reaction by-products, the reaction temperature is preferably at least about 50° C., more preferably about 60° C. to about 90° C. and most preferably from about 70° C. to about 80° C. After the coupling reaction is completed to the desired extent, the mixture is cooled and phases are separated. The reaction mixture is concentrated and an organic solvent such as heptane is added. Purification of the 2′,6′-dichloro-5-fluoro-2-methoxy-biphenyl may be carried out for example by washing with water, contacting the resulting mixture with silica gel followed by filtration. The 2′,6′-dichloro-5-fluoro-2-methoxy-biphenyl may be concentrated as described below and used directly for Step 2a. The reaction yield of the Step 1a is preferably about 88-92%.

In Step 2a of Scheme X, the 2′,6′-dichloro-5-fluoro-2-methoxy-biphenyl is brominated using a brominating agent in the presence of an organic acid. In an embodiment, N-bromosuccinimide (NBS) is used as the brominating agent and para-toluenesulfonic acid (PTSA) is used as the organic acid. A reaction solvent may also be used, for example, acetic acid or formic acid.

In one embodiment of step 2a, a solution of 2′,6′-dichloro-5-fluoro-2-methoxy-biphenyl is concentrated under vacuum. Optionally a solvent such as acetic acid or formic acid is added and removed as a chase to further purify the intermediate. To the 2′,6′-dichloro-5-fluoro-2-methoxy-biphenyl is added N-bromosuccinimide (NBS), para-toluenesulfonic acid (pTSA) and acetic acid. The amount of NBS is preferably from about 1 eq to about 1.5 eq. based on the moles of 2′,6′-dichloro-5-fluoro-2-methoxy-biphenyl. The amount of pTSA is preferably from about 0.1 eq to about 0.5 eq. based on the moles of 2′,6′-dichloro-5-fluoro-2-methoxybiphenyl. The suspension is preferably heated to about 50° C. to about 55° C. and stirred for about 24 hours. The reaction is quenched with an aqueous solution of sodium metabisulfite and the product is collected by filtration. The yield of the bromination is preferably about 88% to about 92%. The overall yield of Steps 1a and 2a is preferably about 77% to about 85%. In another embodiment, formic acid could be used in place of acetic acid. The resulting 3-bromo-2′,6′-dichloro-5-fluoro-2-methoxy-biphenyl can be used directly for Step 3a or alternatively can be recrystallized from a suitable solvent such as a mixture of acetic acid and water or heptanes.

In Step 3a(i) of Scheme X, 3-bromo-2′,6′-dichloro-5-fluoro-2-methoxybiphenyl is reacted with a Grignard reagent to form a compound of formula C-2 where Y is 5-fluorine, Z is chlorine at the 2- and 6-positions, R¹ is methyl, and X′ is Cl and/or Br. In an embodiment, the Grignard reagent is formed by adding dropwise isopropyl magnesium chloride in tetrahydrofuran (THF) solution to a cold (e.g., about −6 to −3° C.) solution of 3-bromo-2′,6′-dichloro-5-fluoro-2-methoxy-biphenyl in THF. In an embodiment, the resulting solution stirred at about −3 to about 2° C. for about 2 to about 3 hours.

In step 3a(ii) of Scheme X, the resulting Grignard reagent is reacted with (2S)-(+)-glycidyl tosylate in the presence of a copper catalyst. In an embodiment, the Grignard reagent of formula C-2 is preferably cooled to about −25 to about −30° C., and CuI is added. Preferably, CuI is added in an amount of about 0.01 eq. to about 0.1 eq based on the 3-bromo-2′,6′-dichloro-5-fluoro-2-methoxybiphenyl. The resulting mixture may be stirred for example for about 30 minutes to about 45 minutes in this temperature range. A solution of (2S)-(+)-glycidyl tosylate in THF is added dropwise to the mixture. The (2S)-(+)-glycidyl tosylate is preferably added in an amount of from about 1 equivalents to about 1.5 equivalents based on the 3-bromo-2′,6′-dichloro-5-fluoro-2-methoxy-biphenyl. Preferably during this step, the reaction temperature is maintained at about −29° C. to about −21° C. Following addition of the (2S)-(+)-glycidyl tosylate, the reaction mixture may be stirred, for example, for an additional 3 to 4 hours. Upon completion of the reaction, the reaction may be quenched with, for example, an ammonium chloride solution. The organic layer is separated and preferably is further washed, extracted with a solvent such as toluene, and concentrated to obtain one or more of the following Grignard reaction product intermediates as an oil:

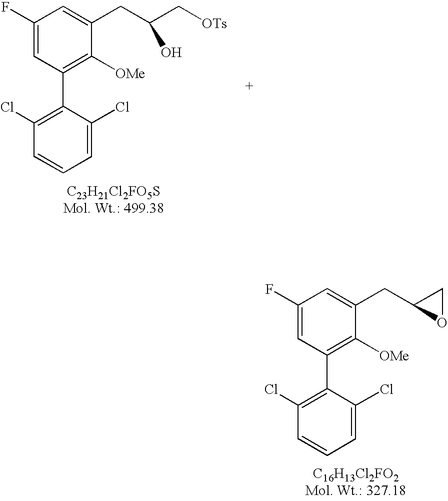

One of ordinary skill in the art would appreciate that, unlike in Scheme I, it is not necessary to contact the resulting Grignard reaction product intermediate with NaOH as shown in Example 3 to convert the hydroxytosylate to the corresponding epoxide. Instead, the above Grignard reaction product intermediates may be reacted directly in Step 4a, i.e. without isolation.

In step 4a of Scheme X above, the Grignard reaction product intermediates are contacted with potassium phthalimide in the presence of a solvent to form (2S)-3-[5-fluoro-3-(2,6-dichlorophenyl)-2-methoxyphenyl]-1-N-phthalimidopropan-2-ol.

In an embodiment, the Grignard reaction product intermediates are dissolved in N,N-dimethyl formamide (DMF) and contacted with potassium phthalimide. In one embodiment, about 1 to about 2 equivalents of potassium phthalimide is used based on starting bromo-2′,6′-dichloro-5-fluoro-2-methoxy-biphenyl. In another embodiment, the contacting is carried out at a temperature of about 80° C. to about 86° C. The contact time is preferably about 8 to about 10 hours. The resulting crude (2S)-3-[5-fluoro-3-(2,6-dichlorophenyl)-2-methoxyphenyl]-1-N-phthalimidopropan-2-ol may be purified using techniques such as washing the organic layer with suitable solvents, distillation and recrystallization. Preferably the yield based on starting bromo-2′,6′-dichloro-5-fluoro-2-methoxy-biphenyl yield is about 57% to about 67%.

In step 5a of Scheme X, (2S)-3-[5-fluoro-3-(2,6-dichlorophenyl)-2-methoxyphenyl]-1-N-phthalimidopropan-2-ol undergoes alcohol mesylation to form (2S)-3-[3-(2,6-dichlorophenyl)-5-fluoro-2-methoxyphenyl]-1-N-phthalimidopropan-2-yl methanesulfonate. In an embodiment, to a solution of (2S)-3-[5-fluoro-3-(2,6-dichlorophenyl)-2-methoxyphenyl]-1-N-phthalimidopropan-2-ol in THF is added triethylamine (TEA) followed by dropwise addition of methanesulfonyl chloride (MsCl). In one embodiment, the TEA is added in an amount of from about 1 to about 1.5 equivalents based on (2S)-3-[5-fluoro-3-(2,6-dichlorophenyl)-2-methoxyphenyl]-1-N-phthalimidopropan-2-ol starting material. In another embodiment, the MsCl is added in an amount of from about 1 to about 1.5 equivalents based on (2S)-3-[5-fluoro-3-(2,6-dichlorophenyl)-2-methoxyphenyl]-1-N-phthalimidopropan-2-ol starting material. In certain embodiments, the TEA and MsCl are both added in an amount of about 1.5 equivalents. In another embodiment, instead of using THF, other solvents such as dichloromethane and acetonitrile may be used. The reaction is preferably carried out at room temperature until completion, for example from about 1 to about 2 hours. After the reaction is completed, water is added to the mixture. Preferably, the resulting white suspension is stirred at room temperature for about 2 hours. The (2S)-3-[3-(2,6-dichlorophenyl)-5-fluoro-2-methoxyphenyl]-1-N-phthalimidopropan-2-yl methanesulfonate may be isolated by filtration. Preferably the (2S)-3-[3-(2,6-dichlorophenyl)-5-fluoro-2-methoxyphenyl]-1-N-phthalimidopropan-2-yl methanesulfonate is produced in a yield of about 95%. Optionally, the (2S)-3-[3-(2,6-dichlorophenyl)-5-fluoro-2-methoxyphenyl]-1-N-phthalimidopropan-2-yl methanesulfonate may be recrystallized again in solvents such as THF or mixtures of solvents such as THF and water. However, the enantiomeric excess may decrease, for example, from 90% to 80% after one recrystallization.

In Step 6a, (2S)-3-[3-(2,6-dichlorophenyl)-5-fluoro-2-methoxyphenyl]-1-N-phthalimidopropan-2-yl methanesulfonate is subjected to a ring closing reaction using a methyl ether cleavage to form 2R-7-(2,6-dichlorophenyl)-5-fluoro-2,3-dihydro-2-(N-phthalimidomethyl)benzofuran. In an embodiment, to a stirring suspension of (2S)-3-[3-(2,6-dichlorophenyl)-5-fluoro-2-methoxyphenyl]-1-N-phthalimidopropan-2-yl methanesulfonate in toluene is added boron tribromide (BBr₃). Preferably, BBr₃ is added in an amount of about 1 eq. to about 1.5 eq. based on (2S)-3-[3-(2,6-dichlorophenyl)-5-fluoro-2-methoxyphenyl]-1-N-phthalimidopropan-2-yl methanesulfonate starting material. The reaction mixture is preferably stirred at a reaction temperature of about 18° C. to about 22° C. until the desired conversion of starting material has been reached. Preferably such a conversion is reached in about 18 hours or less. In an alternative embodiment, dichloromethane may be used instead of toluene. In such an embodiment the reaction temperature may be from about −78° C. to about room temperature (e.g., about 23° C.). Following completion of the reaction, the 2R-7-(2,6-dichlorophenyl)-5-fluoro-2,3-dihydro-2-(N-phthalimidomethyl)benzofuran may be isolated for example by cooling the mixture to about 0° C. to about 5° C. and adding methanol to form a suspension. The suspension may be concentrated and additional methanol may be added for purifying and filtering the 2R-7-(2,6-dichlorophenyl)-5-fluoro-2,3-dihydro-2-(N-phthalimidomethyl)benzofuran. Optionally, the product may be recrystallized again in solvents such as toluene or mixtures of solvents such as toluene and heptanes. The enantiomeric excess may improve for example from 93% to over 99% after one or two recrystallizations.

In Step 7a of Scheme X, the phthalimide protecting group is removed from 2R-7-(2,6-dichlorophenyl)-5-fluoro-2,3-dihydro-2-(N-phthalimidomethyl)benzofuran to form 2R-7-(2,6-dichlorophenyl)-5-fluoro-2,3-dihydro-2-aminomethylbenzofuran.

In an embodiment, to a stirring suspension of 2R-7-(2,6-dichlorophenyl)-5-fluoro-2,3-dihydro-2-(N-phthalimidomethyl)benzofuran in a solvent mixture of ethanol and water, hydrazine hydrate is added. In one embodiment, the volume ratio of ethanol to water is from about 4:1 to about 3:2. In another embodiment, the hydrazine hydrate is added in an amount of about 2 eq. to about 5 eq. based on 2R-7-(2,6-dichlorophenyl)-5-fluoro-2,3-dihydro-2-(N-phthalimidomethyl)benzofuran starting material. Preferably, the reaction mixture is heated to reflux and stirred until the desired conversion of starting material is reached. In one embodiment, the reaction time is about 2 hours. Following completion of the reaction, the resulting 2R-7-(2,6-dichlorophenyl)-5-fluoro-2,3-dihydro-2-aminomethylbenzofurane may be isolated by adding water and t-butylmethyl ether (TBME) to the reaction mixture and separating the phases and washing the aqueous with TBME. The combined organic layers are washed with 1% sodium hydroxide followed by water.

In Step 8a of Scheme X, the crude 2R-7-(2,6-dichlorophenyl)-5-fluoro-2,3-dihydro-2-aminomethylbenzofuran is converted to a hydrochloride salt and purified. In an embodiment, the crude 2R-7-(2,6-dichlorophenyl)-5-fluoro-2,3-dihydro-2-aminomethylbenzofuran in TBME is concentrated under reduced pressure and TBME is replaced by isopropanol. To the 2R-7-(2,6-dichlorophenyl)-5-fluoro-2,3-dihydro-2-aminomethylbenzofuran solution is added a solution of hydrochloric acid in isopropanol (IPA). Preferably the hydrochloric acid is present in an amount of about 1 to about 1.5 equivalents based on the molar amount of 2R-7-(2,6-dichlorophenyl)-5-fluoro-2,3-dihydro-2-aminomethylbenzofuran. To the IPA suspension is added water. The mixture is preferably heated to about 75° C. to about 78° C. to give a clear solution. This solution is gradually cooled and concentrated. The product is isolated by filtration. Preferably, the overall yield based on the starting amine is about 82% to about 92%. The enantiomeric excess may improve for example from 97.8% in the starting material to 98.6% in the hydrochoride salt. Optionally, the product may be recrystallized again in solvents such as IPA or mixtures of solvents such as IPA and water.

The resulting 2R-7-(2,6-dichlorophenyl)-5-fluoro-2,3-dihydro-2-aminomethylbenzofuran hydrochloride in some embodiments form needle shaped crystals. These crystals may be milled as shown in Step 9a if desired to aid in further processing.

In other embodiments of the present invention, the process of the present invention provides compositions containing (2R)-7-(2,6-dichlorophenyl)-5-fluoro-2,3-dihydro-2-aminomethylbenzofuran hydrochloride. In some embodiments, the compositions contain (2R)-7-(2,6-dichlorophenyl)-5-fluoro-2,3-dihydro-2-aminomethylbenzofuran hydrochloride in an amount of at least about 97, 97.5, 98, 98.5, 99, 99.5, 99.8 weight percent where the percentages are based on the total weight of the composition.

In some other embodiments, the composition containing (2R)-7-(2,6-dichlorophenyl)-5-fluoro-2,3-dihydro-2-aminomethylbenzofuran hydrochloride contains from about 9.5 weight percent to about 11.6 weight percent HCl as measured by ion chromatography based on the total weight of the composition. In other embodiments, the composition containing (2R)-7-(2,6-dichlorophenyl)-5-fluoro-2,3-dihydro-2-aminomethylbenzofuran hydrochloride contains from about 10 weight percent to about 10.5 weight percent HCl as measured by ion chromatography based on the total weight of the composition.

In other embodiments, the composition containing (2R)-7-(2,6-dichlorophenyl)-5-fluoro-2,3-dihydro-2-aminomethylbenzofuran hydrochloride preferably contains no more than about 2.0 area percent HPLC of total organic impurities and more preferably no more than about 1.5 area percent HPLC total organic impurities relative to the total area of the HPLC chromatogram. In other embodiments, the composition containing 2R-7-(2,6-dichlorophenyl)-5-fluoro-2,3-dihydro-2-aminomethylbenzofuran hydrochloride preferably contains no more than about 0.6 area percent HPLC of any single impurity and more preferably no more than about 0.5 area percent HPLC of any single impurity relative to the total area of the HPLC chromatogram. According to another embodiment, the composition containing 2R-7-(2,6-dichlorophenyl)-5-fluoro-2,3-dihydro-2-aminomethylbenzofuran hydrochloride preferably contains no more than about 0.2 area percent HPLC of any single impurity relative to the total area of the HPLC chromatogram. According to yet another embodiment, the composition containing 2R-7-(2,6-dichlorophenyl)-5-fluoro-2,3-dihydro-2-aminomethylbenzofuran hydrochloride preferably contains no more than about 0.2 area percent HPLC of total impurities relative to the total area of the HPLC chromatogram.

In yet other embodiments of the invention, the composition containing (2R)-7-(2,6-dichlorophenyl)-5-fluoro-2,3-dihydro-2-aminomethylbenzofuran hydrochloride preferably contains no more than about the following residual solvents individually alone or in any combination: 0.5 weight percent THF, 0.5 weight percent ethanol, 0.5 weight percent isopropylacetate, 0.5 weight percent heptane, 0.5 weight percent hexanes, 0.5 weight percent isopropanol and/or 0.5 weight percent t-butyl methyl ether based on the total weight of the composition. In other embodiments, the composition containing (2R)-7-(2,6-dichlorophenyl)-5-fluoro-2,3-dihydro-2-aminomethylbenzofuran hydrochloride preferably contains no more than about 0.03 weight percent to about 0.04 weight percent ethanol and/or 0.04 weight percent to 0.05 weight percent isopropanol.

In other embodiments, the present invention is directed to intermediates. In certain other embodiments compositions comprising each of the intermediates and one or more organic impurities and/or one or more residual solvents are provided. Examples of intermediates include:

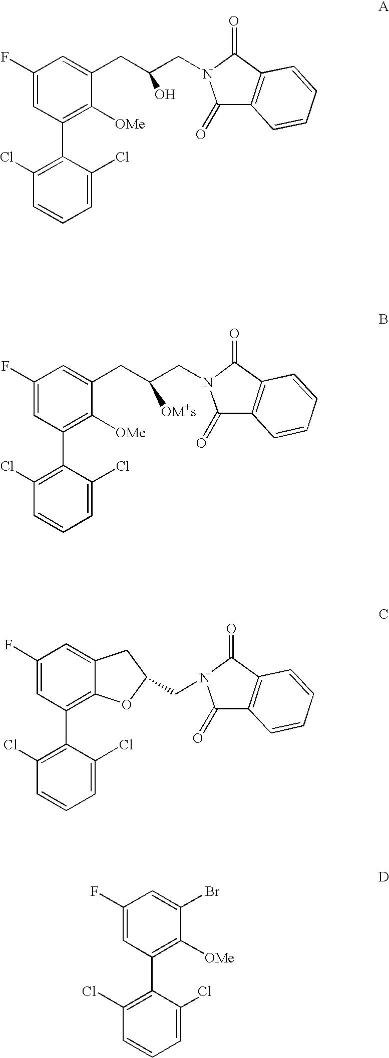

### EXPERIMENTAL

Another method for the synthesis of 2R-(−)-7-(2,6-dichlorophenyl)-5-fluoro-2,3-dihydro-2-aminomethylbenzofuran hydrochloride is illustrated Scheme XI and more fully described in the following examples:

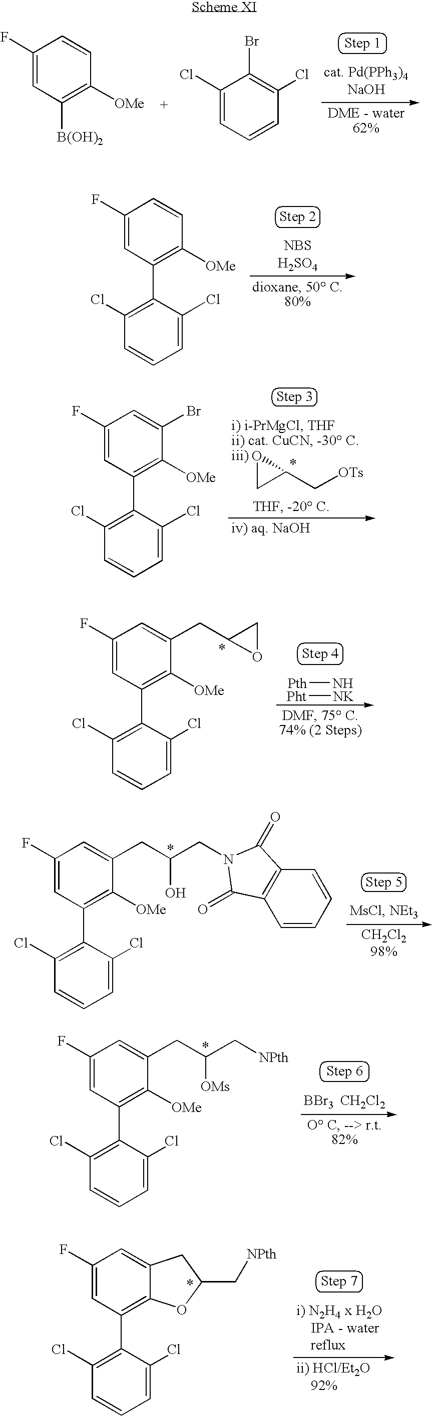

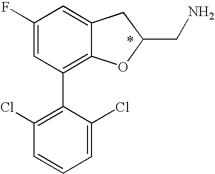

### Example 1

### 1-Methoxy-4-fluoro-2′,6′-dichlorobiphenyl

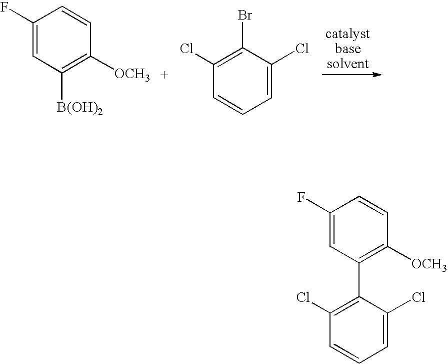

To a solution of NaOH (54 g, 1.35 mol) in water (400 mL) heated to 60° C. was added dimethoxyethane (400 mL), then dichlorobromobenzene (Aldrich, 60 g, 0.267 mol) and boronic acid (50 g, 0.294 mol). To the resulting stirred emulsion, solid Pd(PPh₃)₄ (9.5 g, 8.2 mmol) was added and washed down with 100 mL of DME. The greenish mixture was heated at reflux (ca. 80° C.) while being stirred mechanically. The course of reaction was monitored by HPLC. After 2 hr, 9.0 g (0.053 mol) of additional boronic acid and 2.0 g (1.7 mmol) of the catalyst were added to the reaction mixture and the heating was continued for 16 hr longer. More boronic acid (5.8 g, 0.034 mol) and the catalyst (0.5 g, 0.4 mmol) were added at that point and the mixture was kept at reflux for 7 hr longer (23 hr was total reaction time).

The heating was stopped and 600 mL of heptane and 300 mL of water were added. The mixture was allowed to cool to room temperature and then was filtered through Celite. The layers were separated, the organic layer was washed with water, three times with brine, dried with MgSO₄ and filtered through a pad of Magnesol. The clear colorless solution was concentrated on a rotary evaporator to a colorless oil (weight 72 g). The oil was triturated with 120 mL of heptane which caused crystallization of a white solid. The mixture was left in a refrigerator overnight, the separated crystals were filtered and dried in air. Yield 51 g, 93% pure. The major impurity was determined to be the homo-coupling product 13. Additional recrystallization of the material from heptane gave crystals of 98% purity. Yield 45 g (62%) as white crystals.

### Example 2

### 1-Methoxy-2-bromo-4-fluoro-2′,6′-dichlorobiphenyl

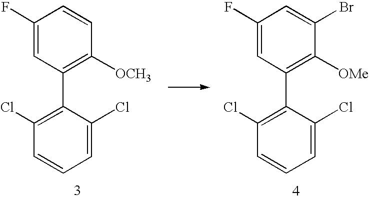

To a magnetically stirred solution of the biarene 3 (38.0 g, 0.140 mol) in 190 mL of dioxane placed into a 500-mL round-bottom flask equipped with a temperature probe, conc. sulfuric acid (38 mL) was added slowly. To the warm solution, solid NBS (26.7 g, 0.150 mol) was added in one portion (no exothermic heating was observed here). The resulting solution was heated in a mantle at 50° C. The reaction progress monitored by HPLC. After 18 hr, only trace amount of the starting arene was detected.

The reaction mixture was allowed to cool to r.t., then it was poured onto 400 g of ice. Heptane (100 mL) was added and the mixture was transferred to the separatory funnel. The aqueous layer was separated and extracted with additional portions of heptane (2×100 mL). Combined organic solutions were washed once with water (30 mL), then aq. Na₂S₂O₃ solution, and, finally, with 1 M aq. NaOH solution (2×30 mL). Light-yellow clear organic solution was dried with MgSO₄, filtered through a cotton plug and evaporated in vacuum. The resulting yellow oil was re-dissolved in 55 mL of heptane.

The first batch of crystals (25.5 g) slowly separated from the heptane solution at r.t. and was filtered and dried in air. Purity 98% (HPLC at 215 nm), white crystals. M.p. 67-69° C.

The second batch of the product (13.9 g) was isolated from the mother liquor by chilling it in a dry-ice-acetone bath, filtering off the precipitated solid and drying it in a vacuum desiccator over CaSO₄. Purity 97% (HPLC area % at 215 nm), white amorphous powder. M.p. 47-56° C. Total yield 39.4 g (80%). ¹H NMR (300 MHz, CDCl₃) δ: 7.42 (m, J=8.1 Hz, 2H), 7.39 (dd, J=3.0, 7.7 Hz, 1H), 7.30 (dd, J=8.1 Hz, 1H), 6.86 (dd, J=3.0, 8.0 Hz, 1H), 3.56 (s, 3H).

### Example 3

### 2-[5-Fluoro-3-(2,6-dichlorophenyl-2-methoxybenzyl]oxirane

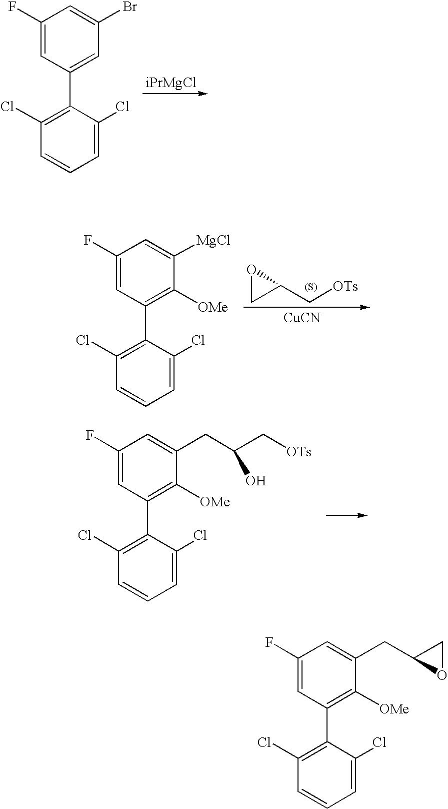 Generation of the Grignard Reagent.

Aryl bromide (25.0 g, 71.4 mmol) was placed into a 500-mL flask equipped with a magnetic stirrer, nitrogen inlet, temperature probe and a rubber septum. The flask was purged excessively with nitrogen, then left under positive nitrogen pressure. Dry THF (100 mL) was transferred into the flask via a syringe. The solution was chilled in an ice bath to 2° C.

A solution of i-PrMgCl in THF (1.9 M, Aldrich, 39.5 mL, 75 mmol) was added slowly to the solution in the flask via a syringe (20 minutes addition time, the temperature was maintained between 2 and 6° C.). The resulting yellowish solution was left in the bath for 18 hr allowing it to reach room temperature.

Reaction with Glycidyl Tosylate.

The solution of the Grignard reagent was chilled to −30° C. by placing the flask in a bath with partially frozen dichloroethane (M.p. −45° C.). CuCN (0.45 g, 5.0 mmol, 7 mol %; Aldrich) was added to the flask via syringe as a slurry in dry THF. The resulting mixture was stirred for 1 hr at −30° C., then (S)-(+)-glycidyl tosylate (15.5 g, 68 mmol, Aldrich) dissolved in 10 mL of dry THF was added to the solution (addition time 30 minutes, reaction mixture temperature was maintained between −22 and −29° C.). The reaction was left stirring at −31° C. for 2 hr, then the DCE bath was replaced with a partially frozen o-xylene bath. Over the next 3 hours the temperature was allowed to reach −18° C. HPLC analysis of the quenched aliquot showed complete disappearance of glycidyl tosylate.

To the cold reaction mixture, 100 mL of aq. NH₄Cl solution (prepared by 1:1 dilution of the saturated solution with water) was added. The phases were separated. The aqueous layer was extracted with 50 mL of MTBE. Combined organic solutions were washed with 30 mL of brine.

Closure to the Epoxide.

To the solution of the intermediate hydroxytosylate was added aq. solution of NaOH prepared by mixing 20 mL of 10 M stock solution (200 mmol) with 30 mL of water. The resulting bi-phasic mixture was stirred rapidly with a magnetic stirrer so that the mixture was broken into fine emulsion. After 18 hr at room temp. (checked by HPLC) the mixture was transferred to a separatory funnel and the phases were separated. The aqueous phase was extracted with 100 mL of MTBE, combined organic solutions were washed with brine and dried with MgSO₄. After filtration through a paper filter, light-yellow solution was evaporated in vacuum to give a mixture the epoxide and des-bromo-arene as a light-yellow oil which solidified upon cooling to room temp. Weight 23.06 g. The mixture was used in the subsequent step without purification.

### Example 4

### 2S-3-[5-Fluoro-3-(2,6-dichlorophenyl)-2-methoxyphenyl]-1-N-phthalimidopropan-2-ol

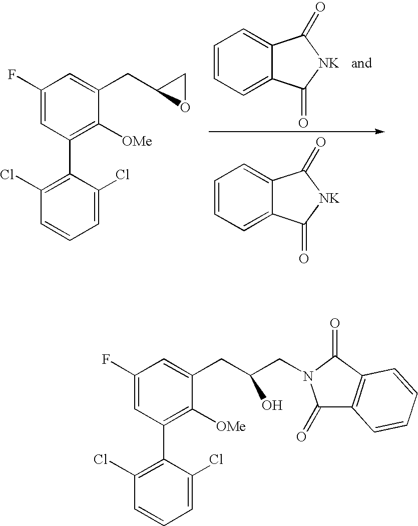

The epoxide (22.6 g of the crude mixture from the previous step, ca. 67 mmol), phthalimide (10.3 g, 70 mmol) and its potassium salt (12.9 g, 70 mmol) were placed in a 250 round-bottom flask equipped with a magnetic stirrer, a nitrogen inlet, and a temperature probe. Dry DMF (100 mL) was added to the mixture. The reaction flask was briefly purged with nitrogen and then was being heated at 75° C. with stirring for 20 hr (the progress was monitored by HPLC). Once no starting epoxide was detected, the mixture was allowed to cool to room temp. and then mixed with 200 mL of ice-water slush. The product was extracted with MTBE (2×100 mL). The organic solution was washed with solution prepared from 2 parts of 1 M aq. NaOH, 3 parts brine, and 5 parts water (2×100 mL), then with brine until neutral pH. The resulting organic solution was dried with MgSO₄, filtered through a paper filter and evaporated in vacuum. The product started to crystallize during the evaporation. The volume of the solvent was reduced to ca. 40 mL, then the residue was triturated with 200 mL of hexanes. The white solid was filtered, washed with hexanes and dried in air. Yield 23.25 g (74% over 3 steps, based on the amount of glycidyl tosylate). M.p. 165-168° C. ¹H NMR (300 MHz, CDCl₃) δ: 7.86 (m, 2H), 7.72 (m, 2H), 7.43 (m, 1H), 7.41 (m, 1H), 7.27 (m, 1H), 7.08 (dd, J=3.0, 8.8 Hz, 1H), 6.79 (dd, J=3.0 Hz, 8.1 Hz, 1H), 4.23 (d⁵, J=3.3, 4.3, 5.7, 7.9, 8.5 Hz, 1H), 3.85 (dd, J=3.3, 14.1 Hz, 1H), 3.80 (dd, J=8.5, 14.1 Hz, 1H), 3.42 (s, 3H), 2.96 (dd, J=4.3, 13.9 Hz, 1H), 2.92 (dd, J=7.9, 13.9 Hz, 1H), 2.80 (d, J=5.7 Hz, 1H). ES MS, m/z: 474 (M+H)⁺, Cl₂ isotope pattern. Analytical purity: 97% (HPLC area % at 215 nm).

### Example 5

### 2S-3-[5-Fluoro-3-(2,6-dichlorophenyl)-2-methoxyphenyl]-1-N-phthalimidopropan-2-yl methanesulfonate

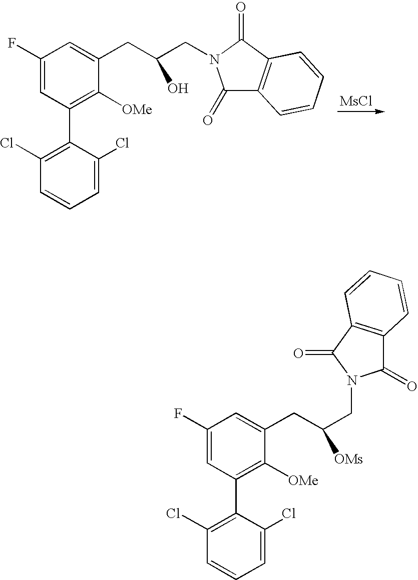

In a 500 mL Erlenmeyer flask equipped with a magnetic stirrer, temperature probe and an addition funnel was placed the product of Example 4 (22.0 g, 46.4 mmol), CH₂CL₂ (200 mL) and triethylamine (9.7 mL, 70 mmol). Into the addition funnel was placed CH₂Cl₂ (20 mL) and methanesulfonyl chloride (5.4 mL, 70 mL). The solution of MsCl was added dropwise (addition time 10 minutes) to the stirred solution in the flask. The reaction mixture was allowed to stir at room temp. for 2 hr (checked by HPLC). White solid separated from the solution over that time.

Water (100 mL) was added to the reaction mixture while stirring it rapidly. About 120 mL of DCM was distilled off on a rotary evaporator. The residue was triturated with 200 mL of hexanes. The solid was filtered and washed excessively with water and hexanes. The cake was dried on the filter for I hr then overnight in a vacuum desiccator over CaSO₄. Yield 25.2 g (98%) as a white fluffy crystals. M.p. >200° C. (decomp.)

¹H NMR (300 MHz, CDCl₃) δ: 7.86 (m, 2H), 7.72 (m, 2H), 7.43 (m, 2H), 7.29 (m, 1H), 7.09 (dd, J=3.1, 8.5 Hz, 1H), 6.82 (dd, J=3.1, 8.3 Hz, 1H), 5.28 (m, 1H), 4.09 (dd, J=8.6, 14.6 Hz, 1H), 3.90 (dd, J=3.3, 14.6 Hz, 1H), 3.45 (s, 3H), 3.18 (dd, J=5.4, 14.0 Hz, 1H), 3.09 (dd, J=7.8, 14.0 Hz, 1H), 2.65 (s, 3H). ¹³C NMR (100 MHz, dmso-d₆) δ: 167.6, 157.6 (d, J=242 Hz), 152.4 (d, J=2 Hz), 134.8, 134.4, 134.3 (d, J=16 Hz), 131.6, 131.4 (d, J=20 Hz), 131.4, 130.8, 128.3, 123.1, 118.7 (d, J=22 Hz), 116.7 (d, J=24 Hz), 78.5, 60.5, 40.8, 37.6, 33.2. ES MS, m/z: 552 (M+H)⁺, Cl₂ isotope pattern. Analytical purity 99.6% (HPLC area % at 215 nm).

### Example 6

### 2R-7-(2,6-Dichlorophenyl)-5-fluoro-2,3-dihydro-2-(N-phthalimidomethyl)benzofuran

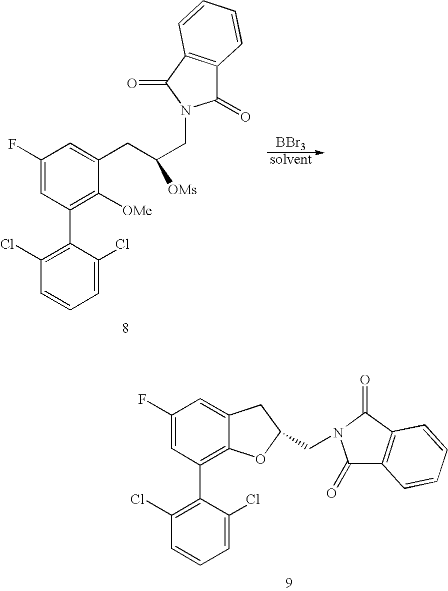

The product of Example 5, 2S-3-[5-fluoro-3-(2,6-dichlorophenyl)-2-methoxyphenyl]-1-N-phthalimidopropan-2-yl methanesulfonate, (22.1 g, 40.0 mmol) and dichloromethane (200 mL) were placed into a 500-mL flask equipped with a magnetic stirrer, a temperature probe, a nitrogen inlet and a 50-mL addition funnel. The flask and the addition funnel were purged briefly with nitrogen. The slurry in the flask was chilled in an ice bath to 4° C. A 1 M solution of BBr₃ in CH₂Cl₂ (Aldrich, 42 mL, 42 mmol) was placed into the addition funnel and was added dropwise to the stirred contents of the flask. The stirring was continued allowing the temperature of the reaction mixture to reach 16° C. over 2-hr period and then at room temp. for 3 hr longer.

The reaction mixture was quenched by slowly pouring it into the solution prepared from NaHCO₃ (11 g, 131 mmol) and 200 mL of water. After 30 minutes of stirring, the layers were separated. Aqueous layer was extracted with dichloromethane (2×50 mL). Combined organic solutions were washed with 100 mL of water, then dried with MgSO₄. The drying agent was filtered off and washed with ethyl acetate. The volume of the filtrate was reduced to about 50 mL on rotary evaporator. The product separated as white or light-yellow solid. The slurry was triturated with 40 mL of a 50:50 hexanes-MTBE mixture, the solid was filtered, washed with the above mixture of solvents and dried on the filter.

Yield 14.4 g (82%) as a light-yellow solid. M.p. 222.5-224.5° C.

¹H NMR (400 MHz, dmso-d₆) δ: 7.85 (m, 4H), 7.53 (m, 2H), 7.41 (m, 1H), 7.19 (dd, J=2.7, 8.2 Hz, 1H), 6.86 (dd, J=2.7, 9.3 Hz, 1H), 5.09 (m, 1H), 3.79 (m, 2H), 3.43 (dd, J=9.3, 16.6 Hz, 1H), 3.15 (dd, J=5.9, 16.6 Hz, 1H). ¹³C NMR (100 MHz, dmso-d₆) δ: 167.8, 156.4 (d, J=237 Hz), 152.2, 134.5, 134.4 (d, J=30 Hz), 133.5, 131.5, 130.6, 128.6 (d, J=9.4 Hz), 128.1 (d, J=3.6 Hz), 123.1, 118.2, 118.1, 114.9 (d, J=9.3 Hz), 112.9 (d, J=25 Hz), 80.0, 41.1, 33.2. ES MS, m/z: 442 MH⁺, Cl₂ isotope pattern. Analytical purity: 99.9% (HPLC area % at 215 nm).

### Example 7

### 2R-7-(2,6-Dichlorophenyl)-5-fluoro-2,3-dihydro-2-aminomethylbenzofuran hydrochloride

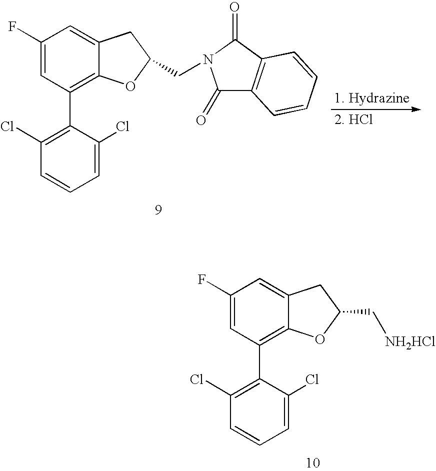

The product of Example 6, 2R-7-(2,6-dichlorophenyl)-5-fluoro-2,3-dihydro-2-(N-phthalimidomethyl)benzofuran (12.9 g, 29.2 mmol) was mixed with 70 mL of isopropanol and 15 mL of water. Hydrazine hydrate (55% hydrazine content, Aldrich, 5 mL, 90 mmol) was then added and the reaction mixture was magnetically stirred and heated at gentle reflux for 2 hr. Dissolution of the staring material and formation of a clear solution was an indication that the reaction was done.

To the hot solution was added 40 mL of 1 M aqueous NaOH and 100 mL of water. The product was extracted with MTBE (3×50 mL). Combined extracts were washed with 60 mL of 0.2 M aq. NaOH, then with water (2×50 mL) and finally with brine (50 mL). Resulting clear solution was dried over Na₂SO₄ for 1 hr, filtered through a paper filter and evaporated in vacuum to afford a light-yellow oil.

The oil was dissolved in 50 mL of EtOAc and to the solution was added rapidly 2 M solution of HCl in diethyl ether (Aldrich, 15 mL, 30 mmol). The salt precipitated rapidly. It was slurred with 100 mL of ether, then the slurry was stirred for 30 minutes in an ice bath. The salt was filtered, washed with 100 mL of ether, dried first on the filter in the stream of air until the filter reached room temp. and then overnight in a vacuum desiccator over CaSO₄. Yield 9.4 g (92%) as white crystals. M.p. 231-233° C. [α]_{D}²⁵=−18.2 (MeOH, 1 wt %). ¹H NMR (400 MHz, dmso-d₆) δ: 8.25 (broad s, 3H), 7.57 (m, J=8.1 Hz, 2H), 7.45 (dd, J=8.1 Hz, 1H), 7.24 (dd, J=2.6, 8.1 Hz, 1H), 6.90 (dd, J=2.6, 9.6 Hz, 1H), 5.05 (d⁴, J=9.2, 7.9, 7.0, 4.5 Hz, 1H), 3.45 (dd, J=9.2, 16.6 Hz, 1H), 3.17 (dd, J=7.0, 16.6 Hz), 3.10 (dd, J=13.4, 4.5 Hz, 1H), 3.04 (dd, J=13.4, 7.9 Hz, 1H).

¹³C NMR (400 MHz, dmso-d₆) δ: 156.4 (d, J=257 Hz), 151.9, 134.5, 134.2, 133.5, 130.5, 128.7 (d, J=11 Hz), 128.2 (d, J=21 Hz), 118.3 (d, J=9 Hz), 115.0 (d, J=25 Hz), 112.9 (d, J=25 Hz), 80.0, 42.1, 32.8. ES MS, m/z: 312 (M+H), Cl₂ isotope pattern.

Enantiomeric purity: 99.4% ee (chiral HPLC on Chiracel OD-H 0.46×25 cm, 1 mL/minutes 90% heptane/DIEA, 10% ethanol, area % at 280 nm). Analytical purity: 99.8% (HPLC on Prodigy ODS3 0.46×15 cm, 1 mL/min water/TFA−MeCN/TFA 100 min gradient 0-100%, area % at 215 nm). Seventeen impurities in the range of 0.003-0.06 area % were detected totaling 0.19%. For C₁₅H₁₃Cl₃FNO found C 51.59%, H 3.81%, N 3.87%, anionic Cl 10.49%; calc'd C 51.68%, H 3.76%, N 4.02%, anionic Cl 10.17%.

### Example 8

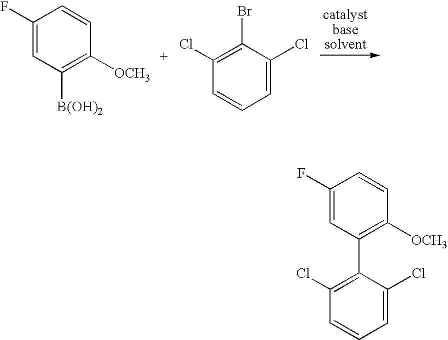

To a stirring 70° C. solution of 2,6-dichlorobromobenzene (0.6 Kg, 2.656 mol), boronic acid (0.588 Kg, 3.46 mol) and palladium tetrakis triphenylphosphine (0.0614 Kg, 0.053 mol) in dimethoxyethane (6.0 L) is added an aqueous solution of sodium hydroxide (0.53 Kg, 13.25 mol in 3.0 Kg water). The mixture is refluxed for 18 hours. The mixture is cooled and phases are separated. The reaction mixture is concentrated and heptanes is added. The solution is washed with water. To the product solution is added silica gel The suspension is stirred for 2 hours then filtered. The intermediate product 2′,6′-dichloro-5-fluoro-2-methoxy-biphenyl in solution in heptanes is concentrated and is used directly for the bromination step. The reaction yield of the Suzuki coupling is (88-92%).

### Example 9

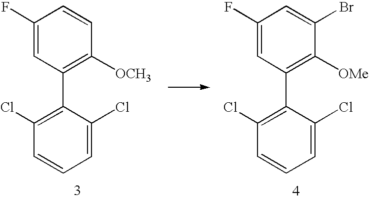

The solution of the intermediate obtained from Example 8 is stripped under vacuum and acetic acid is used for the chase. To the residue is added N-bromosuccinimide (0.575 Kg), para-toluenesulfonic acid (0.086 Kg) and acetic acid (3.35 L). The suspension is heated to (50-55° C.) and stirred for 24 hours. The reaction is quenched with an aqueous solution of sodium metabisulfite and the product is collected by filtration. The yield of the bromination is (88-92%). The overall yield is (77-85%). The crude product can used directly for subsequent step or can be recrystallized from acetic acid and water or heptanes.

### Example 10

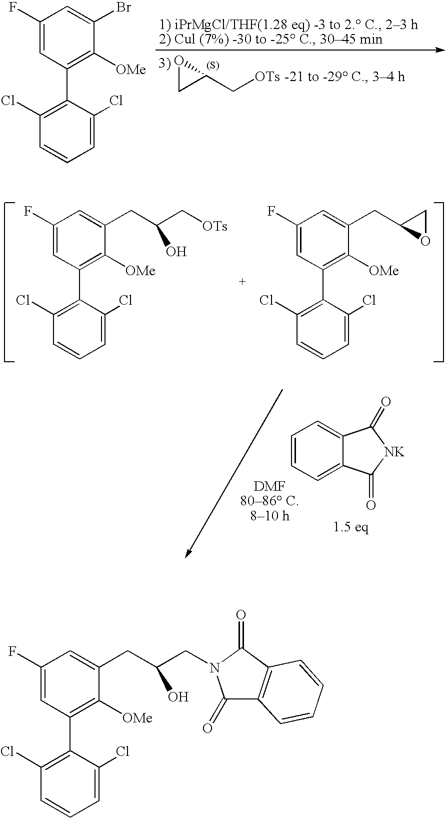

The Grignard reagent is generated by adding dropwise the 2.0 M isopropyl magnesium chloride (1.04 Kg, 2.14 mol) in THF (2.0 M) to a cold (pre-cooled to −6 to −3° C.) solution of the bromide from Example 9 in THF (2.64 L), followed by further stirring at −3 to 2° C. for 2 to 3 hours to complete the formation of Grignard reagent. Cool the Grignard reagent to −25 to −30° C., add a catalytical amount of CuI (25.1 g, 0.132 mol), stir the mixture at this temperature range for 30-45 minutes. To the cold reaction mixture is added dropwise a solution of (2S)-(+)-glycidyl tosylate (0.409 Kg, 1.70 mol) in THF (0.413 L) and keep the reaction temperature at between −29 to −21° C. during the addition, stir the mixture for another 3-4 hours at this temperature range. The cold reaction mixture is quenched with 15% ammonium chloride solution, then followed by the proper work up to give the Grignard reaction product as an oil. This product is dissolved in DMF and stirred with potassium phthalimide (0.523 Kg, 2.82 mol, 1.5 equiv) at 80 to 86° C. for 8-10 h, and worked up to afford the crude phthalimide-protected compound, which is recrystallized from iPrOAc-heptanes.

### Example 11

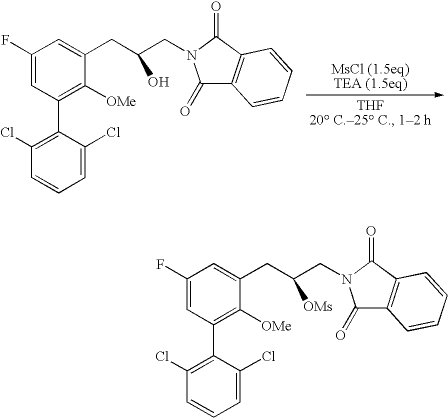

To a stirring solution of (2S)-3-[3-(2,6-dichlorophenyl)-5-fluoro-2-methoxyphenyl]-1-N-phthalimidopropan-2-ol (0.374 Kg, 0.789 mol) in THF (1.75 L) is added triethylamine (0.1194 Kg, 1.18 mol) followed by dropwise addition of methanesulfonyl chloride (0.1352 Kg, 1.18 mol). The mixture is stirred at room temperature for 1-2 hours. After water (1.87 L) is added to the mixture, the white suspension is stirred at room temperature for 2 hour. The product was collected by filtration. The product is provided in 95.0% yield, at 98.5% strength, 1.06% total impurities, 0.47% single largest impurity, enantiomeric excess at 98.7%, throughput is 18.0% (R), 10.4% (W).

### Example 12

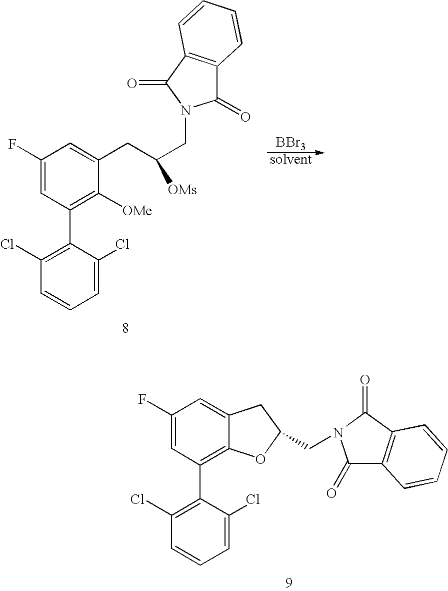

To a stirring (18-22° C.) suspension of starting material (400 g, 0.724 mol) in toluene is added boron tribromide (200 g, 0.797 mol). The reaction mixture is stirred for 18 hours at (18-22° C.). The reaction mixture is cooled to (0-5° C.) and to the mixture is added methanol. The suspension is concentrated by reduced pressure distillation and methanol is added to the residue. The suspension is heated to reflux then cooled to (0-5° C.), and stirred for 1 hour. The product is collected by filtration. The yield is (80-85%).

### Example 13

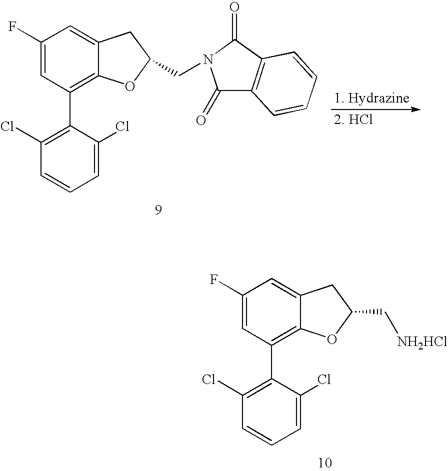

To a stirring (18-22° C.) suspension of 2R-7-(2,6-dichlorophenyl)-5-fluoro-2,3-dihydro-2-(N-phthalimidomethyl)benzofuran (370 g, 0.837 mol) in ethanol:water (3:2) is added hydrazine hydrate (126 g, 2.51 mol, 3 equiv.). The reaction mixture is heated to reflux and stirred for 2 hours. To the reaction mixture is added water (1.110 L) and TBME (1.110 L). The phases are separated and aqueous are washed with TBME. The product solution is washed with 1% sodium hydroxide followed by water.

The crude amine in TBME is concentrated under reduced pressure and TBME is replaced by isopropanol. To the amine solution is added a solution of hydrochloric acid in IPA (1.2 equi). To the suspension is added 3.8% water and the mixture is heated to give a clear solution at (75-78° C.). This solution is cooled to 65° and seeded, stirred at 65° for 30 minutes then cooled to 55° C. and stirred for 1 hour. The suspension is cooled to (30-35° C.), concentrated under vacuum then cooled to −10° C. The product is isolated by filtration. The overall yield is (82-92%). The enantiomeric excess improves from 97.8% in the starting material to 98.6%. The product can be recrystallize from IPA/water improving the enantiomeric excess to 98.9% with a recovery of 91.5%.

### Example 14

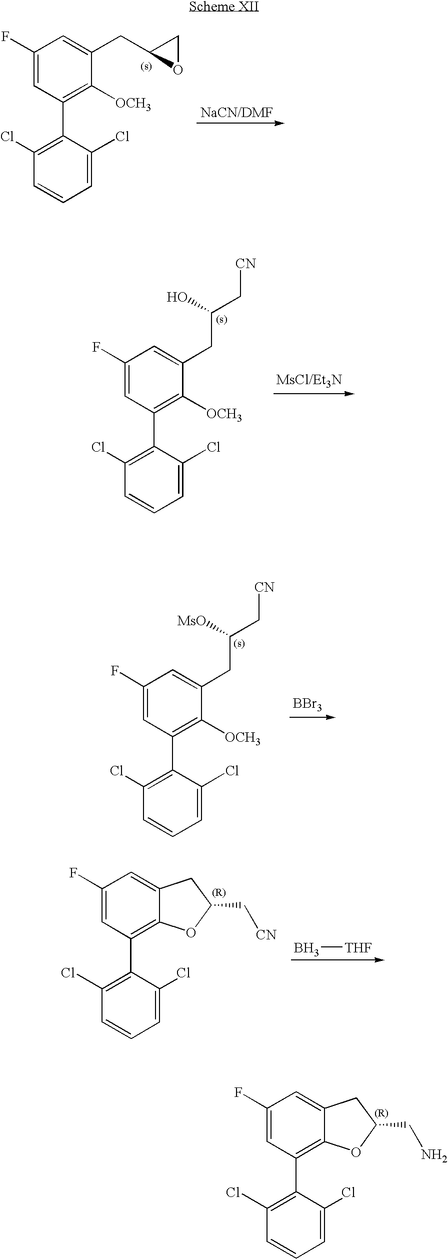

### Example 14a

### (S)-4-(2′,6′-Dichloro-5-fluoro-2-methoxybiphenyl-3-yl)-3-hydroxybutanenitrile

To a solution of (S)-2-[(2′,6′-dichloro-5-fluoro-2-methoxybiphenyl-3-yl)-methyl]oxirane (2.28 g, 7.0 mmol) and sodium cyanide (1.7 g, 34.8 mmol) in DMF (90 ml) and water (15 ml) was heated at 70° C. for 18 hour. The reaction was quenched with water. The mixture was extracted with methylene chloride. The organic layer was washed with water and dried over sodium sulfate. The organic solvent was removed under vacuum. Chromatography with 10-40% ethyl acetate in hexanes afforded product 1.65 g (66%) as a light yellow oil. MS ESI m/e 371.1 (M+NH₄)⁺; [α]=+5.0° (1% solution in MeOH).

### Example 14b

### (S)-1-Cyano-3-(2′6′-dichloro-5-fluoro-2-methoxybiphenyl-3-yl)propan-2-yl-methanesulfonate

To a solution of (S)-4-(2′,6′-dichloro-5-fluoro-2-methoxybiphenyl-3-yl)-3-hydroxybutanenitrile (2.0 g, 5.6 mmol) in methylene chloride (20 ml) was added methanesulfonyl chloride (1.48 ml, 8.4 mmol) and triethylamine (1.2 ml, 8.4 mmol) at 0° C. The resulting mixture was stirred for 2 hours at room temperature. The reaction was quenched with water. The mixture was extracted with methylene chloride. The organic layer was washed with water and dried over sodium sulfate. The organic solvent was removed under vacuum. Chromatography with 0-40% ethyl acetate in hexanes afforded product 1.77 g (72.5%) as a clear oil. MS ESI m/e 449.0 (M+NH₄)⁺; [α]=−19.0° (1% solution in MeOH).

### Example 14c

### (R)-2-[7-(2,6-Dichlorophenyl)-5-fluoro-2,3-dihydrobenzofu ran-2-yl]acetonitrile

To a solution of (S)-1-cyano-3-(2′6′-dichloro-5-fluoro-2-methoxybiphenyl-3-yl)propan-2-ylmethanesulfonate (1.77 g, 4.1 mmol) in methylene chloride was added boron tribromide (0.58 ml, 6.2 mmol) at −78° C. The resulting mixture was stirred at −78° C. to 15° C. for overnight. The mixture was poured into ice and extracted with methylene chloride. The organic layer was washed with water and dried over sodium sulfate. The organic solvent was removed under vacuum. Chromatography with 0-30% ethyl acetate in hexanes afforded product 1.07 g (81%) as a clear oil. MS EI m/e 321 M⁺; [α]=−35.0° (1% solution in MeOH).

### Example 14d

### (R)-2-[7-(2,6-Dichlorophenyl)-5-fluoro-2,3-dihydrobenzofuran-2-yl]ethanamine

To a solution of (R)-2-[7-(2,6-dichlorophenyl)-5-fluoro-2,3-dihydrobenzofuran-2-yl]acetonitrile (1.0 g, 3.1 mmol) was added borane-tetrahydrofuran complex (1.0 M solution in THF, 15.5 mmol, 16.5 ml) at room temperature. The resulting mixture was refluxed for 3 hours. The reaction was quenched with saturated ammonium chloride and extracted with methylene chloride. The organic layer was washed with water. The solvent was removed under vacuum. Chromatography with 0-10% methanol in methylene chloride plus 1% ammonium hydroxide afforded a clear oil. The oil was dissolved in ethyl acetate and made into its hydrochloric salt using excess ethereal hydrochloric acid to give a white solid 0.56 g: mp. 238-240° C. [α]=+49.0° (1% solution in MeOH). Elemental Analysis for: C₁₆H₁₄FCl₂NO.1HCl.0.6H₂O Theory: C, 51.46 H, 4.37 N, 3.75; Found: C, 51.19 H, 3.94 N, 3.56

All patents, publications, and other documents cited herein are hereby incorporated by reference in their entirety.

We claim:

1. A method for preparing a compound of formula I-a: 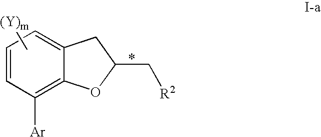
wherein:
m is 0-3;
R² is CN, N₃, or N(R³)(R⁴);
R³ and R⁴ are each independently hydrogen, an amine protecting group, C₁₋₆ alkyl, lower haloalkyl, 3-6 membered cycloaliphatic, or alkylcycloaliphatic, or R³ and R⁴ are taken together with the nitrogen to which they are attached to form a cyclic amine protecting group or a 3-6 membered saturated or partially unsaturated ring having 1-2 heteroatoms independently selected from nitrogen, oxygen, or sulfur;
each Y is independently hydrogen, chlorine, fluorine, CN, —OH, C₁₋₈ alkyl, C₁₋₆ perfluoroalkyl, C₁₋₆ alkoxy, C₁₋₆ perfluoroalkoxy, 6-10 membered aryl, 6-10 membered aryloxy, 5-10 membered heteroaryl having 1 to 4 heteroatoms independently selected from nitrogen, oxygen or sulfur, C₂₋₈ alkenyl, C₁₋₆ alkanesulfonamido, dialkylamino of 1 to 6 carbon atoms per alkyl moiety, C₃₋₈ cycloaliphatic, or 3-8 membered heterocycloalkyl having 1 to 3 heteroatoms independently selected from nitrogen, oxygen or sulfur; and
Ar is an optionally substituted 6-10 membered aryl or 5-10 membered heteroaryl having 1-4 heteroatoms independently selected from nitrogen, oxygen, or sulfur,
comprising the steps of:
providing a compound of the formula F-1: 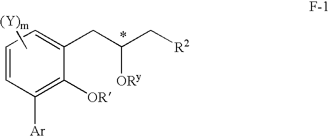
wherein:
m is 0-3;
R¹ is hydrogen or a suitable hydroxyl protecting group;
OR^{y} is a suitable leaving group;
R² is CN, N₃, or N(R³)(R⁴);
R³ and R⁴ are each independently hydrogen, an amine protecting group, C₁₋₆ alkyl, lower haloalkyl, 3-6 membered cycloaliphatic, or alkylcycloaliphatic, or R³ and R⁴ are taken together with the nitrogen to which they are attached to form a cyclic amine protecting group or a 3-6 membered saturated or partially unsaturated ring having 1-2 heteroatoms independently selected from nitrogen, oxygen, or sulfur;
each Y is independently hydrogen, chlorine, fluorine, CN, —OH, C₁₋₈ alkyl, C₁₋₆ perfluoroalkyl, C₁₋₆ alkoxy, C₁₋₆ perfluoroalkoxy, 6-10 membered aryl, 6-10 membered aryloxy, 5-10 membered heteroaryl having 1 to 4 heteroatoms independently selected from nitrogen, oxygen or sulfur, C₂₋₈ alkenyl, C₁₋₆ alkanesulfonamido, dialkylamino of 1 to 6 carbon atoms per alkyl moiety, C₃₋₈ cycloaliphatic, or 3-8 membered heterocycloalkyl having 1 to 3 heteroatoms independently selected from nitrogen, oxygen or sulfur; and
Ar is an optionally substituted 6-10 membered aryl or 5-10 membered heteroaryl having 1-4 heteroatoms independently selected from nitrogen, oxygen, or sulfur, and
converting the compound of formula F-1 to a compound of formula I-a or a
pharmaceutically acceptable salt thereof.

2. The process of claim 1 wherein Ar is 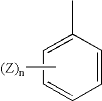
wherein:
n is 0-5; and
each Z is independently Cl, F, CN, —OH, cyano, C₁₋₆ alkyl, C₁₋₆ perfluoroalkyl, C₁₋₆ alkoxy, or C₁₋₆ perfluoroalkoxy.

3. The process of claim 2 further comprising the steps of:
(a) providing a compound of formula C-1: 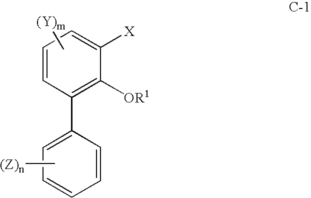
wherein:
m is 0-3;
n is 0-5;
X is halogen;
R¹ is a suitable hydroxyl protecting group;
each Y is independently hydrogen, chlorine, fluorine, CN, —OH, C₁₋₈ alkyl, C₁₋₆ perfluoroalkyl, C₁₋₆ alkoxy, C₁₋₆ perfluoroalkoxy, 6-10 membered aryl, 6-10 membered aryloxy, 5-10 membered heteroaryl having 1 to 4 heteroatoms independently selected from nitrogen, oxygen or sulfur, C₂₋₈ alkenyl, C₁₋₆ alkanesulfonamido, dialkylamino of 1 to 6 carbon atoms per alkyl moiety, C₃₋₈ cycloaliphatic, or 3-8 membered heterocycloalkyl having 1 to 3 heteroatoms independently selected from nitrogen, oxygen or sulfur; and
each Z is independently Cl, F, CN, —OH, cyano, C₁₋₆ alkyl, C₁₋₆ perfluoroalkyl, C₁₋₆ alkoxy, or C₁₋₆ perfluoroalkoxy,
(b) converting the compound of formula C-1 to a compound of formula C-2: 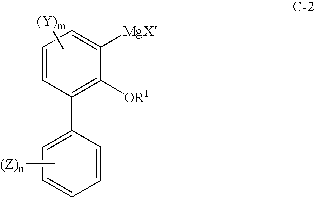
wherein:
m is 0-3;
n is 0-5;
X′ is halogen;
R¹ is a suitable hydroxyl protecting group;
each Y is independently hydrogen, chlorine, fluorine, CN, —OH, C₁₋₈ alkyl, C₁₋₆ perfluoroalkyl, C₁₋₆ alkoxy, C₁₋₆ perfluoroalkoxy, 6-10 membered aryl, 6-10 membered aryloxy, 5-10 membered heteroaryl having 1 to 4 heteroatoms independently selected from nitrogen, oxygen or sulfur, C₂₋₈ alkenyl, C₁₋₆ alkanesulfonamido, dialkylamino of 1 to 6 carbon atoms per alkyl moiety, C₃₋₈ cycloaliphatic, or 3-8 membered heterocycloalkyl having 1 to 3 heteroatoms independently selected from nitrogen, oxygen or sulfur; and
each Z is independently Cl, F, CN, —OH, cyano, C₁₋₆ alkyl, C₁₋₆ perfluoroalkyl, C₁₋₆ alkoxy, or C₁₋₆ perfluoroalkoxy,
(c) reacting the compound of formula C-2 with a chiral non-racemic epoxide of the formula: 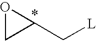
wherein L is a leaving group, to produce a compound of formula D-1: 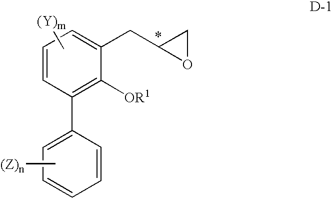
wherein:
m is 0-3;
n is 0-5;
R¹ is a suitable hydroxyl protecting group;
each Y is independently hydrogen, chlorine, fluorine, CN, —OH, C₁₋₈ alkyl, C₁₋₆ perfluoroalkyl, C₁₋₆ alkoxy, C₁₋₆ perfluoroalkoxy, 6-10 membered aryl, 6-10 membered aryloxy, 5-10 membered heteroaryl having 1 to 4 heteroatoms independently selected from nitrogen, oxygen or sulfur, C₂₋₈ alkenyl, C₁₋₆ alkanesulfonamido, dialkylamino of 1 to 6 carbon atoms per alkyl moiety, C₃₋₈ cycloaliphatic, or 3-8 membered heterocycloalkyl having 1 to 3 heteroatoms independently selected from nitrogen, oxygen or sulfur; and
each Z is independently Cl, F, CN, —OH, cyano, C₁₋₆ alkyl, C₁₋₆ perfluoroalkyl, C₁₋₆ alkoxy, or C₁₋₆ perfluoroalkoxy,
and
(d) and converting the compound of formula D-1 to a compound of formula E-1.

4. The process of claim 2 further comprising the steps of:
(a) providing a compound of the formula: 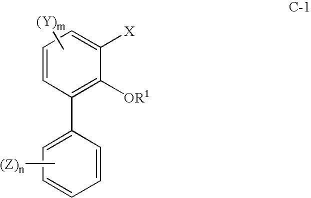
wherein:
m is 0-3;
n is 0-5;
X is halogen;
R¹ is a suitable hydroxyl protecting group;
each Y is independently hydrogen, chlorine, fluorine, CN, —OH, C₁₋₈ alkyl, C₁₋₆ perfluoroalkyl, C₁₋₆ alkoxy, C₁₋₆ perfluoroalkoxy, 6-10 membered aryl, 6-10 membered aryloxy, 5-10 membered heteroaryl having 1 to 4 heteroatoms independently selected from nitrogen, oxygen or sulfur, C₂₋₈ alkenyl, C₁₋₆ alkanesulfonamido, dialkylamino of 1 to 6 carbon atoms per alkyl moiety, C₃₋₈ cycloaliphatic, or 3-8 membered heterocycloalkyl having 1 to 3 heteroatoms independently selected from nitrogen, oxygen or sulfur; and
each Z is independently Cl, F, CN, —OH, cyano, C₁₋₆ alkyl, C₁₋₆ perfluoroalkyl, C₁₋₆ alkoxy, or C₁₋₆ perfluoroalkoxy,
(b) converting the compound of formula C-1 to a compound of formula C-2: 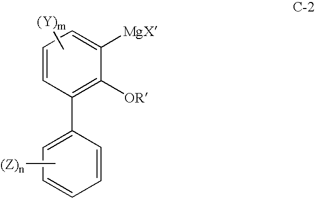
wherein:
m is 0-3;
n is 0-5;
X′ is halogen;
R¹ is a suitable hydroxyl protecting group;
each Y is independently hydrogen, chlorine, fluorine, CN, —OH, C₁₋₈ alkyl, C₁₋₆ perfluoroalkyl, C₁₋₆ alkoxy, C₁₋₆ perfluoroalkoxy, 6-10 membered aryl, 6-10 membered aryloxy, 5-10 membered heteroaryl having 1 to 4 heteroatoms independently selected from nitrogen, oxygen or sulfur, C₂₋₈ alkenyl, C₁₋₆ alkanesulfonamido, dialkylamino of 1 to 6 carbon atoms per alkyl moiety, C₃₋₈ cycloaliphatic, or 3-8 membered heterocycloalkyl having 1 to 3 heteroatoms independently selected from nitrogen, oxygen or sulfur; and
each Z is independently Cl, F, CN, —OH, cyano, C₁₋₆ alkyl, C₁₋₆ perfluoroalkyl, C₁₋₆ alkoxy, or C₁₋₆ perfluoroalkoxy,
(c) reacting the compound of formula C-2 with a chiral non-racemic epoxide of formula 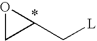
wherein L is a leaving group, to produce at least one compound of formula: 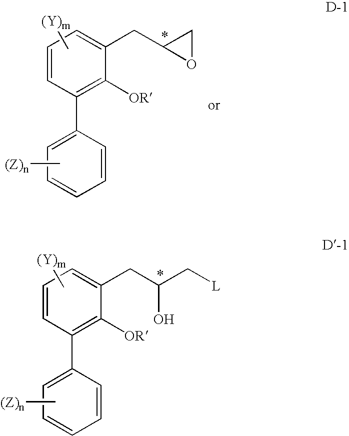
and
(d) converting the compound of formula D-1 or D′-1 to a compound of formula E-1.

5. The process of claim 4 further comprising the steps of:
(a) providing a compound of the formula: 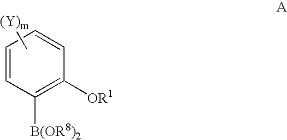
wherein:
m is 0-3;
R¹ is hydrogen or a suitable hydroxyl protecting group;
each Y is independently hydrogen, chlorine, fluorine, CN, —OH, C₁₋₈ alkyl, C₁₋₆ perfluoroalkyl, C₁₋₆ alkoxy, C₁₋₆ perfluoroalkoxy, 6-10 membered aryl, 6-10 membered aryloxy, 5-10 membered heteroaryl having 1 to 4 heteroatoms independently selected from nitrogen, oxygen or sulfur, C₂₋₈ alkenyl, C₁₋₆ alkanesulfonamido, dialkylamino of 1 to 6 carbon atoms per alkyl moiety, C₃₋₈ cycloaliphatic, or 3-8 membered heterocycloalkyl having 1 to 3 heteroatoms independently selected from nitrogen, oxygen or sulfur; and
each R⁸ is independently hydrogen or C₁₋₆ alkyl,
(b) contacting the compound of formula A with a compound of formula A-1 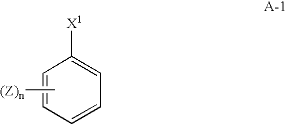
wherein:
n is 0-5;
each Z is independently Cl, F, CN, —OH, cyano, C₁₋₆ alkyl, C₁₋₆ perfluoroalkyl, C₁₋₆ alkoxy, or C₁₋₆ perfluoroalkoxy; and
X¹ is Cl, Br, I, triflate (—OSO₂CF₃) or other perfluoroalkylsulfonate,
in the presence of a palladium catalyst to produce a compound of the formula B-1: 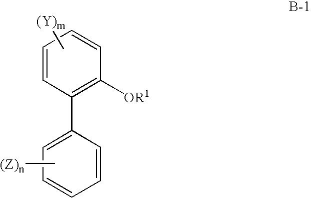
wherein:
m is 0-3;
n is 0-5;
R¹ is hydrogen or a suitable hydroxyl protecting group;
each Y is independently hydrogen, chlorine, fluorine, CN, —OH, C₁₋₈ alkyl, C₁₋₆ perfluoroalkyl, C₁₋₆ alkoxy, C₁₋₆ perfluoroalkoxy, 6-10 membered aryl, 6-10 membered aryloxy, 5-10 membered heteroaryl having 1 to 4 heteroatoms independently selected from nitrogen, oxygen or sulfur, C₂₋₈ alkenyl, C₁₋₆ alkanesulfonamido, dialkylamino of 1 to 6 carbon atoms per alkyl moiety, C₃₋₈ cycloaliphatic, or 3-8 membered heterocycloalkyl having 1 to 3 heteroatoms independently selected from nitrogen, oxygen or sulfur; and
each Z is independently Cl, F, CN, —OH, cyano, C₁₋₆ alkyl, C₁₋₆ perfluoroalkyl, C₁₋₆ alkoxy, or C₁₋₆ perfluoroalkoxy,
and
(c) contacting the compound of formula B-1 with a halogenating agent to produce a compound of formula C-1: 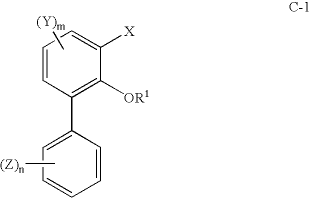
wherein:
m is 0-3;
n is 0-5;
X is halogen;
R¹ is a suitable hydroxyl protecting group;
each Y is independently hydrogen, chlorine, fluorine, CN, —OH, C₁₋₈ alkyl, C₁₋₆ perfluoroalkyl, C₁₋₆ alkoxy, C₁₋₆ perfluoroalkoxy, 6-10 membered aryl, 6-10 membered aryloxy, 5-10 membered heteroaryl having 1 to 4 heteroatoms independently selected from nitrogen, oxygen or sulfur, C₂₋₈ alkenyl, C₁₋₆ alkanesulfonamido, dialkylamino of 1 to 6 carbon atoms per alkyl moiety, C₃₋₈ cycloaliphatic, or 3-8 membered heterocycloalkyl having 1 to 3 heteroatoms independently selected from nitrogen, oxygen or sulfur; and
each Z is independently Cl, F, CN, —OH, cyano, C₁₋₆ alkyl, C₁₋₆ perfluoroalkyl, C₁₋₆ alkoxy, or C₁₋₆ perfluoroalkoxy.

6. The process of claim 5 wherein R¹ is C₁₋₄ alkyl.

7. The process of claim 5 wherein X is Br.

8. The process of claim 3 wherein conversion of the compound of formula C-1 to the compound of formula D-1 comprises:
(a) contacting the compound of formula C-1 with a Grignard reagent to form a compound of formula C-2: 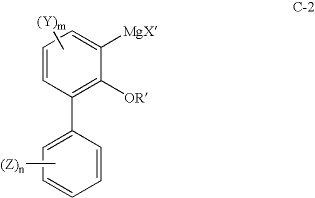
wherein:
m is 0-3;
n is 0-5;
X′ is halogen;
R¹ is a suitable hydroxyl protecting group;
each Y is independently hydrogen, chlorine, fluorine, CN, —OH, C₁₋₈ alkyl, C₁₋₆ perfluoroalkyl, C₁₋₆ alkoxy, C₁₋₆ perfluoroalkoxy, 6-10 membered aryl, 6-10 membered aryloxy, 5-10 membered heteroaryl having 1 to 4 heteroatoms independently selected from nitrogen, oxygen or sulfur, C₂₋₈ alkenyl, C₁₋₆ alkanesulfonamido, dialkylamino of 1 to 6 carbon atoms per alkyl moiety, C₃₋₈ cycloaliphatic, or 3-8 membered heterocycloalkyl having 1 to 3 heteroatoms independently selected from nitrogen, oxygen or sulfur; and
each Z is independently Cl, F, CN, —OH, cyano, C₁₋₆ alkyl, C₁₋₆ perfluoroalkyl, C₁₋₆ alkoxy, or C₁₋₆ perfluoroalkoxy,
(b) contacting the compound of formula C-2 with a chiral non-racemic epoxide of formula 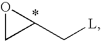
wherein L is a leaving group, to form a compound of the formula D′-1: 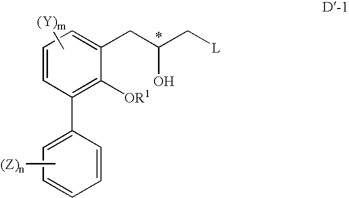
and
(c) contacting the compound of formula D′-1 with a suitable base to produce a compound of formula D-1:

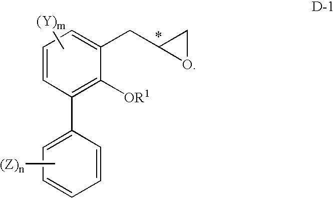

9. The process of claim 8 wherein the contacting of the compound of formula C-2 and 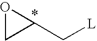
occurs in the presence of a copper salt.

10. The process of claim 8 wherein the contacting of the compound of formula C-2 and 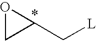
occurs at a temperature of about −15° C. to about −35° C.

11. The process of claim 8 wherein the contacting of the compound of formula C-2 and 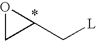
occurs at a temperature of about −25° C. to about −20° C.

12. The process of claim 8 wherein copper salt is at least one of CuCN, Li₂CuCl₄ or CuI.

13. The process of claim 8 wherein the Grignard reagent is isopropylmagnesium chloride.

14. A process for preparing a compound of formula I-a: 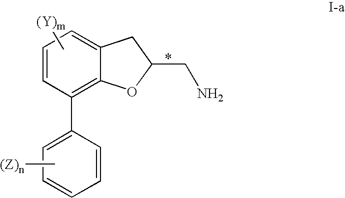
wherein:
m is 0-3;
n is 0-5;
each Y is independently hydrogen, chlorine, fluorine, CN, —OH, C₁₋₈ alkyl, C₁₋₆ perfluoroalkyl, C₁₋₆ alkoxy, C₁₋₆ perfluoroalkoxy, 6-10 membered aryl, 6-10 membered aryloxy, 5-10 membered heteroaryl having 1 to 4 heteroatoms independently selected from nitrogen, oxygen or sulfur, C₂₋₈ alkenyl, C₁₋₆ alkanesulfonamido, dialkylamino of 1 to 6 carbon atoms per alkyl moiety, C₃₋₈ cycloaliphatic, or 3-8 membered heterocycloalkyl having 1 to 3 heteroatoms independently selected from nitrogen, oxygen or sulfur; and
each Z is independently Cl, F, CN, —OH, cyano, C₁₋₆ alkyl, C₁₋₆ perfluoroalkyl, C₁₋₆ alkoxy, or C₁₋₆ perfluoroalkoxy;
comprising the steps of:
(a) contacting a compound of formula E-2: 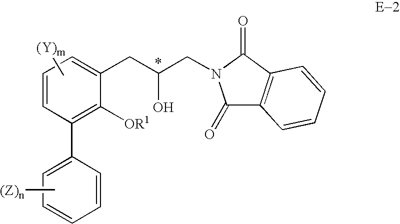
wherein:
m is 0-3;
n is 0-5;
R¹ is a suitable hydroxyl protecting group;
each Y is independently hydrogen, chlorine, fluorine, CN, —OH, C₁₋₈ alkyl, C₁₋₆ perfluoroalkyl, C₁₋₆ alkoxy, C₁₋₆ perfluoroalkoxy, 6-10 membered aryl, 6-10 membered aryloxy, 5-10 membered heteroaryl having 1 to 4 heteroatoms independently selected from nitrogen, oxygen or sulfur, C₂₋₈ alkenyl, C₁₋₆ alkanesulfonamido, dialkylamino of 1 to 6 carbon atoms per alkyl moiety, C₃₋₈ cycloaliphatic, or 3-8 membered heterocycloalkyl having 1 to 3 heteroatoms independently selected from nitrogen, oxygen or sulfur; and
each Z is independently Cl, F, CN, —OH, cyano, C₁₋₆ alkyl, C₁₋₆ perfluoroalkyl, C₁₋₆ alkoxy, or C₁₋₆ perfluoroalkoxy;
with R^{y}Cl and a suitable base to produce a compound of the formula F-1: 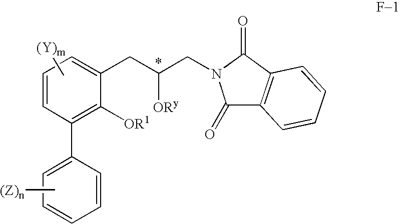
wherein R^{y} is optionally substituted alkylsulphonyloxy, optionally substituted alkenylsulfonyloxy, or optionally substituted arylsulfonyloxy;
(b) contacting the compound of formula F-1 with a methyl ether cleaving agent to produce a compound of formula G-1: 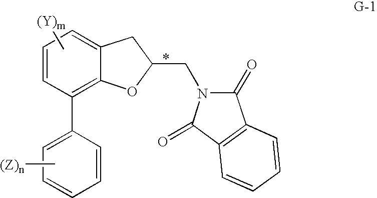
and
(c) converting the compound of formula G-1 to a compound of formula I-a.

15. The process of claim 14 wherein the methyl ether cleaving agent is BBr₃.

16. The process of claim 14 wherein the compound of formula G-1 is converted to a compound of formula I-a in a process comprising contacting the compound of formula G-1 with a hydrazine.

17. The process of claim 16 wherein the hydrazine is hydrazine hydrate or a substituted hydrazine of formula R^{z}NHNH₂, wherein R^{z} is H, C₁₋₆ alkyl, or 5-10 membered aryl.

18. The process of claim 17 wherein the hydrazine contacting step occurs in the presence of an alcohol or THF solvent.

19. The process of claim 2 wherein the compound of formula I-a is 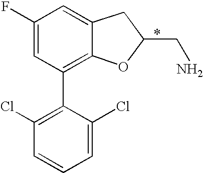
or a pharmaceutically acceptable salt thereof.

20. A method for preparing a compound of formula I′: 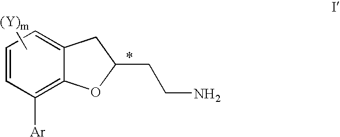
or a pharmaceutically acceptable salt thereof, wherein:
m is 0-3;
each Y is independently hydrogen, chlorine, fluorine, CN, —OH, C₁₋₈ alkyl, C₁₋₆ perfluoroalkyl, C₁₋₆ alkoxy, C₁₋₆ perfluoroalkoxy, 6-10 membered aryl, 6-10 membered aryloxy, 5-10 membered heteroaryl having 1 to 4 heteroatoms independently selected from nitrogen, oxygen or sulfur, C₂₋₈ alkenyl, C₁₋₆ alkanesulfonamido, dialkylamino of 1 to 6 carbon atoms per alkyl moiety, C₃₋₈ cycloaliphatic, or 3-8 membered heterocycloalkyl having 1 to 3 heteroatoms independently selected from nitrogen, oxygen or sulfur; and
Ar is an optionally substituted 6-10 membered aryl or 5-10 membered heteroaryl having 1-4 heteroatoms independently selected from nitrogen, oxygen, or sulfur,
comprising the steps of:
(a) providing a compound of formula D: 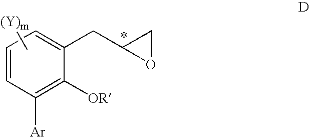
wherein:
m is 0-3;
R¹ is a suitable hydroxyl protecting group;
each Y is independently hydrogen, chlorine, fluorine, CN, —OH, C₁₋₈ alkyl, C₁₋₆ perfluoroalkyl, C₁₋₆ alkoxy, C₁₋₆ perfluoroalkoxy, 6-10 membered aryl, 6-10 membered aryloxy, 5-10 membered heteroaryl having 1 to 4 heteroatoms independently selected from nitrogen, oxygen or sulfur, C₂₋₈ alkenyl, C₁₋₆ alkanesulfonamido, dialkylamino of 1 to 6 carbon atoms per alkyl moiety, C₃₋₈ cycloaliphatic, or 3-8 membered heterocycloalkyl having 1 to 3 heteroatoms independently selected from nitrogen, oxygen or sulfur; and
Ar is an optionally substituted 6-10 membered aryl or 5-10 membered heteroaryl having 1-4 heteroatoms independently selected from nitrogen, oxygen, or sulfur,
(b) treating said compound of formula D with a compound M-CN, wherein M is a suitable metal, to form a compound of formula H: 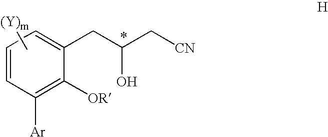
wherein:
m is 0-3;
R¹ is a suitable hydroxyl protecting group;
each Y is independently hydrogen, chlorine, fluorine, CN, —OH, C₁₋₈ alkyl, C₁₋₆ perfluoroalkyl, C₁₋₆ alkoxy, C₁₋₆ perfluoroalkoxy, 6-10 membered aryl, 6-10 membered aryloxy, 5-10 membered heteroaryl having 1 to 4 heteroatoms independently selected from nitrogen, oxygen or sulfur, C₂₋₈ alkenyl, C₁₋₆ alkanesulfonamido, dialkylamino of 1 to 6 carbon atoms per alkyl moiety, C₃₋₈ cycloaliphatic, or 3-8 membered heterocycloalkyl having 1 to 3 heteroatoms independently selected from nitrogen, oxygen or sulfur; and
Ar is an optionally substituted 6-10 membered aryl or 5-10 membered heteroaryl having 1-4 heteroatoms independently selected from nitrogen, oxygen, or sulfur,
(c) converting the hydroxyl group of compound H to a suitable leaving group,
(d) cyclizing compound H to form a compound of formula J: 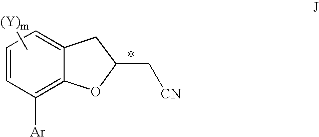
wherein m, Ar, and Y are as defined herein;
and
(e) reducing the cyano group to form a compound of formula I′.
